(19) United States
(12) Patent Application Publication
(10) Pub. No.: US 2003/0028911 A1
(43) Pub. Date: Feb. 6, 2003

(54) Transgenic mammal capable of facilitating production of donor-specific functional immunity

(76) Inventors: Manley Huang, Palo Alto, CA (US); Fiona A. Harding, Santa Clara, CA (US)

Correspondence Address: PENNIE AND EDMONDS, 1155 AVENUE OF THE AMERICAS, NEW YORK, NY, 100362711

(21) Appl. No.: 09/938,689

(22) Filed: Aug. 23, 2001

Related U.S. Application Data
(63) Continuation-in-part of application No. 09/651,361, filed on Aug. 30, 2000.
(60) Provisional application No. 60/151,688, filed on Aug. 31, 1999.

(51) Int. Cl. A01K 67/027

(52) U.S. Cl. 800/18

(57) ABSTRACT

This invention provides for transgenic non-human mammalian models of human disease, methods of making such models as well as methods of using such models to assess efficacy of therapeutic and prophylaxis treatments, to assess the antigenic potential of compounds, and other uses.

The present application is a continuation-in-part of U.S. application Ser. No. 09/651,361 filed Aug. 30, 2000, which claims priority benefits of U.S. Provisional Application Serial No. 60/151,688,filed Aug. 31, 1999, the disclosures of each of which are incorporated by reference herein in their entirety.

This invention was made in part with government support. The government has certain rights in this invention.

### 1. FIELD OF THE INVENTION

The present invention relates to transgenic mammals expressing a plurality of genes from a donor organism allowing for the transgenic mammal to support donor hematopoietic stem cells and facilitate donor-specific functional immunity.

### 2. BACKGROUND OF THE INVENTION

Many human diseases remain incurable in large part due to the lack of an appropriate model system for preclinical studies. Since many diseases are specific to either human pathogens or dysfunctional human tissues, it is difficult to model the course of such afflictions outside of the human body. For example, the basis of allergic responses is deeply rooted in the genetics of the host and cannot be completely studied in a different species. Infectious diseases, such as HIV, have species-specific virulence factors. And cancer cells that arise from a combination of genetic factors usually display altered properties when transplanted into immunodeficient animals.

Unfortunately, there are few methods for directly studying the pathology of human diseases. This in turn limits the development of new drugs and novel therapies. Given the practical and ethical restrictions of experimenting in both humans and higher primates, there is an urgent need to develop alternative models of human diseases.

In models of human disease where an interaction between the disease causing agent and the immune system is suspected, either hematopoietic stem cells or mature circulating lymphocytes are transferred into naturally occurring strains of immunodeficient mice. Although better than their forerunners in certain respects, these models fail to reproduce many of the functional properties of human cells that are critical for unraveling disease processes. On a more basic level, even attempts to transplant hematopoietic stem cells between individuals of the same species have produced allogeneic chimeras that are functionally impaired. The reasons for this are unclear, but involve the inability of the donor stem cells to differentiate properly in the mature lymphoid tissues of the new host.

In an attempt to overcome these problems, researchers have added IL-7, either exogenously or transgenically to mice before engraftment. However, this approach unexpectedly led to further immunological dysfunction. For example, see, Kapp, et al., Blood 92:2024 (1998) (exogenous IL-7 led to decrease in B cell development); Rich, et al., J. Exp. Med. 177:305 (1993) (transgenic IL-7 under the control of immunogloubulin heavy chain promoter and enhancer led to dermal lymphoid infiltration and T and B cell lymphomas); Valenzona, et al., Exp. Hematol. 24:1521 (1996) (IL-7 under the MHC class II promoter induced B lymphoid tumors); Watanabe, et al., J. Exp. Med. 187:389 (1998) (IL-7 transgenic mice developed chronic colitis); Uehira, et al., J. Invest. Dermatol. 110:740 (1998) (IL-7 transgenic mice developed dermatitis);and Mertsching, et al., Eur. J Immunol. 26:28 (1996) (IL-7 transgenic mice developed lymphoproliferative disease).

Thus, there remains a need for a standard transgenic animal model system that supports the functional properties of human (donor) hematopoietic cells. This invention meets this and other needs.

Citation of any reference in this section or any other section of the present specification is not to be construed as an admission that such reference is prior art.

### 3. SUMMARY OF THE INVENTION

The present invention provides for a recipient mammal comprising a disruption in both alleles of a gene such that lymphocyte maturation does not occur and exogenous cytokines. The cytokines are selected from the group consisting of interleukin 3, (IL-3), interleukin-6 (IL-6), interleukin-7 (IL-7), macrophage-colony stimulating factor (M-CSF), granulocyte-colony stimulating factor (GM-CSF), stem cell factor (SCF), leukemia inhibitory factor (LIF) and oncostatin M (OM). In a preferred aspect of this embodiment, the cytokines comprise IL-3, IL-6, IL-7, M-CSF, GM-CSF and SCF. In another preferred aspect of this embodiment, the cytokines are introduced into the mammal transgenically.

In a preferred embodiment, the mammal is a mouse. In another embodiment of the present invention, the mammal is a mouse comprising a disruption in both alleles of a gene such that lymphocyte maturation does not occur; and exogenous (donor specific) transgenes that encode cytokines comprising IL-7, SCF and LIF. In yet another embodiment, the mammal is a mouse comprising a disruption in both alleles of a gene such that lymphocyte maturation does not occur; and exogenous transgenes that encode cytokines comprising GM-CSF, M-CSF and IL-6. In a preferred embodiment, the mammal is a mouse comprising a disruption in both alleles of a gene such that lymphocyte maturation does not occur; and exogenous transgenes that encode cytokines comprising IL-7, SCF, LIF, GM-CSF, M-CSF and IL-6. In each of these embodiments, the disruption is in a gene that modulates VDJ recombination, e.g., a RAG gene. In yet another embodiment, the mammal is a mouse comprising a disruption in both alleles of a gene such that lymphocyte maturation does not occur; and a human transgene comprising a nucleic acid sequence that encodes a MHC Class II DR3 molecule, wherein the transgene comprises naturally linked DRab and DQab alleles.

In another embodiment of this invention, a method of making a mammal with a donor immune system is provided. This method comprises the steps of introducing transgenes into an immunodeficient mammal, wherein the transgenes encode cytokines necessary for the maintenance and maturation of donor-derived cells. In one aspect of this embodiment, the introduction of transgenes is through transfection of embryonic stem cells. In a second aspect of this embodiment, the introduction of transgenes is through pronuclear transfer. In an alternative aspect of this embodiment, the introduction of the transgenes is through breeding the mammal with the transgenes such that the progeny of the mammal will comprise the transgenes.

In a preferred aspect of this embodiment, the mammal is a RAG-1 or a RAG-2 mutant mouse. In another aspect of the invention, the mammal is a RAG-1 or RAG-2 mutant mouse expressing human leukocyte antigen (HLA) Class I and/or Class II genes. In a further aspect of the invention, the mammal is a SCID mouse expressing HLA Class I and/or Class II genes. In yet another aspect of the invention, the mammal is an immunocompetent mouse expressing HLA Class I and/or Class II genes and rendered immunodeficient by, e.g., irradiation conditioning.

In a preferred embodiment, the method comprises inactivating VDJ recombination; and introducing transgenes, wherein said transgenes encode human cytokines necessary for support of human cells in the mouse. In a particular aspect of this embodiment, the mouse is a RAG-1⁻ or a RAG-2⁻ mouse and the mouse further comprises a MHC transgene, e.g., a HLA transgene. In yet another preferred embodiment, the method comprises disrupting both alleles of a gene so that lymphocyte maturation does not occur; inserting a transgene comprising nucleic acid that encodes MHC Class II DR3 and DQ2 molecules, wherein the DRab and DQab alleles are naturally linked; and inactivating murine I-Eα. In another embodiment, the method comprises preventing VDJ recombination by mutating both alleles of the RAG-2 gene; inserting a transgene comprising the Drab and DQab alleles of the MHC Class II DR3 haplotype; and inactivating murine I-Eα.

In yet another embodiment of this invention, a method of determining an immune response to an antigen is provided. Transgenic chimeric mammals are immunized with proteins, peptides, cells or other sources of antigens, to determine epitopes involved in donor cell-derived immune responses. These include, but are not limited to, antigen-specific immunoglobulin production, Tₕₑₗₚₑᵣ responses, T_{cytotoxic} responses, cellular proliferation responses, innate allogeneic or xenogeneic responses, and natural killer cell activity.

3.1 Definitions

Unless defined otherwise, all technical and scientific terms used herein have the same meaning as commonly understood by one of ordinary skill in the art to which this invention belongs. The disclosures of all cited references are incorporated by reference in their entirety. Although any methods and materials similar or equivalent to those described herein can be used in the practice or testing of the present invention, the preferred methods and materials are described. For purposes of the present invention, the following terms are defined below.

The phrase “major histocompatibility complex” (MHC) refers to immune response genes that encode cell surface glycoproteins that regulate interactions among cells of the immune system. The genes were discovered as a result of their involvement in graft rejection. There are two main classes of MHC genes, Class I and Class II. The phrase “human leukocyte antigen” (HLA) refers to the MHC complex of humans. The phrase “MHC restriction” refers to the recognition of peptides by T cells in the context of particular allelic forms of MHC molecules. For a more complete description of the MHC complex in humans, as well as in mice, see, Fundamental Immunology, 4th Ed., Paul (ed.) 1999.

Cells that are “allogeneic” to a mammal are cells that are from an individual of the same species as the mammal but, because of differences in expression of major and minor histocompatibility molecules between the cell donor and the host mammal, are recognized by the host mammal as non-self.

Cells that are “xenogeneic” to a host mammal are cells that are from an individual of a different species as the mammal. Due to significant genetic differences, they are recognized by the host mammal as non-self.

The phrase “bone marrow” refers to the red marrow of the bones of the spine, sternum, ribs, clavicle, scapula, pelvis and skull. This marrow contains hematopoietic stem cells. The phrase “umbilical cord blood” refers to whole blood obtained from the umbilical cord of a newborn. This blood also contains hematopoietic stem cells. The phrase “mobilized peripheral blood” refers to peripheral blood isolated from individuals treated with recombinant growth factors, e.g., granulocyte colony stimulating factor (GM-CSF) and stem cell factor (SCF), for the purpose of increasing the proportion of hematopoietic stem cells in the circulation.

The term “cytokines” refers to proteins that are commonly referred to as cytokines as well as other proteins, such as growth factors, interleukins, immune system modulators, and other types of proteins necessary to maintain an immune system. For example, cytokines encompass the interleukins, stem cell factors, colony stimulating factors and other factors known to those of skill in the art. “Exogenous cytokines” refers to cytokines that are not naturally occurring in the recipient mammal. These cytokines can be species orthologs of naturally occurring cytokines or cytokines that do not have a naturally occurring ortholog in the recipient mammal.

The term “immunodeficiency” refers to a lack of antigen-specific immunity in a mammal. In these mammals, B and T lymphocytes fail to mature properly and are unable to recognize and respond to antigens.

The phrase “recombination activation genes” (RAG) refers to the RAG-I and RAG-2 genes that are involved with initiating the rearrangement of B and T cell antigen receptors. The genetic recombination at the V, D and/or J gene segments is necessary to produce B and T-cell receptors. Mutations in the RAG-1 an RAG-2 genes prevent early steps in this process, and result in a blockade of B cell development in the bone marrow and thymocyte development in the thymus (Mombaerts, et al., Cell 68:869-77 (1992); Shinkai, et al., Cell 68:855-867 (1992)).

The phrase “donor-specific cells with hematopoietic stem cell properties” refer to cells from a donor species that exhibit hematopoietic stem cell properties. The most obvious candidates are hematopoietic stem cells. However, other cells are envisioned, including but not limited to, cells that differentiate into HSC, such as embryonic stem cells.

The phrase “donor immune system” refers to complete or partial immune function that is not naturally found in a recipient mammal. For example, in a recipient mammal of this invention, cytokines necessary for the maintenance of a functional immune system, as well as donor-specific immune cells, are introduced into an immunodeficient mammal, either through introduction of transgenes that encode the cytokines or, less preferably, through the addition of cytokines to the animal. Donor cells are the source of the recipient mammal's immune system (and typically, but not necessarily, the cytokine). It is not necessary that the donor immune system be fully functional, i.e., exhibit all functions of a mammalian immune system found in nature. However, it is preferred that the donor immune system at least comprise donor T and B lymphocytes, and antigen presenting cells such as macrophages and dendritic cells.

The phrase “embryonic stem cells” refers to cells that will grow continuously in culture and retain the ability to differentiate to all cell lineages, including but not limited to, hematopoietic cells. The term “differentiate” or “differentiated” refers to the process of becoming a more specialized cell type. For example, hematopoietic stem cells differentiate into cells of the “lymphoid”, “erythroid” and “myeloid” lineages. Lymphoid cells are cells that mediate the specificity of immune responses. They are divided into two main groups, T and B lymphocytes, and include a small population of large granular lymphocytes, or natural killer cells. Erythroid cells are erythroblasts and erythrocytes. Cells of the myeloid lineage include platelets, neutrophils, basophilic, eosinophils and monocytes.

The phrase “facilitating production of donor-specific functional immunity” refers to the ability of the recipient mammal to develop and maintain a functional donor-derived immune system. Typically, the immune system comprises hematopoietic cells that are specific to the donor as well as cytokines and other ancillary compounds that are necessary, or even desired, to allow the hematopoietic cells to be functional, e.g., bind to antigen, recognize an antigen as foreign or self, communicate with other cells of the immune system so that other cells, e.g., monocytes and macrophages, are activated, or cytokines are released.

The term “introduction” or “introducing” for purposes of this invention refers to the addition of exogenous compounds, particularly cytokine genes, to the recipient mammals of this invention. The compounds can be introduced into the recipient mammals of this invention in a variety of methods, including but not limited to, introduction of the genes that encode the compounds. Introduction of the genes that encode the compounds can be through gene transfer into a non-fetal mammal or transgenically into a gamete or an embryonic mammal. In addition to direct introduction of the genes that encode the compounds, the genetic material can be introduced into a recipient mammal through breeding or cloning, e.g., the introduction of the genes that encode the compounds into the germline of an offspring from a transgenic parent.

The phrase “maintaining an immune system” refers to the ability of exogenous cytokines to support a donor-derived immune system in a recipient mammal that otherwise would not support such an immune system. Typically, however not necessarily, the exogenous cytokines are naturally found in the same species as the donor. Thus, required interactions between the cells of the donor-derived immune system and cytokines naturally found in the donor to maintain the immune system are supplied in the recipient mammal.

The phrase “maintenance and maturation of donor-derived hematopoietic cells” refers to providing cytokines necessary to allow hematopoietic stem cells and other immature cell types to mature into functional cells, e.g., of the immune system, and providing necessary cytokines so that the cells, once mature, survive to function. In addition to the cells of the immune system, the maintenance and maturation of other types of hematopoietic cells, e.g., erythrocytes, platelets, other lymphoid tissue (for example, the gut-associated immune system which consists of Peyer's patches, villi containing intraepithelial lymphocytes, and lymphocytes scattered throughout the lamina propria, and the connective tissue beneath the surface epithelium).

A “mammal” is a warm blooded vertebrate of the class Mammalia, and for the purposes of this invention, excludes humans.

The term “i-mune mouse” refers to a mouse of the present invention, which is immunodeficient and expresses exogenous cytokines.

### 4. BRIEF DESCRIPTION OF THE DRAWINGS

FIGS. 1A-1E demonstrate that allogeneic bone marrow engrafted RAG mice are tolerant to donor and host MHC, but responsive to third party alloantigens. CD4⁺ T cells were isolated by cytotoxic elimination of class II⁺ and CD8⁺ cells from the lymph nodes of a syngeneic engrafted RAG mouse (RAG-(syn), (FIG. 1A); an allogeneic engrafted RAG mouse (RAG-(allo), FIG. 1B); a syngeneic engrafted SCID mouse (SCID-(syn), FIG. 1C); an allogeneic engrafted SCID mouse (SCID-(allo), FIG. 1D); and a RAG mouse, FIG. 1E) engrafted with the same bone marrow preparation as placed in the SCID-(allo) mouse shown in FIG. 1D, as a positive control for the bone marrow inoculum.

CD4⁺ T cells were co-cultured with irradiated LPS-induced splenic blasts from Balb/C (diamonds); C57B1/6 (squares); CBA (circles FIGS. 1A and 1B) or (C57B1/6×CBA)F1 mice (circles, FIGS. 1C, 1D and 1E). Proliferation was assessed colorometrically (Celltiter; Promega) on day (d)=5, and is reported as OD₄₉₀×1000 on the y axis. Background absorption has been subtracted.

FIGS. 2A-2B demonstrate antigen specific IgG responses by RAG-(allo) mice. In FIG. 2A, control Balb/c mice (open triangles), RAG-(allo) mice (closed squares; mouse #30), RAG-(syn) mice (open squares; mouse RN003), and SCID-(allo) mice (closed circles; two animals, SN005 and SN006, are shown) were immunized in the hind foot pads with a total of 50 μg hen egg white lysozyme (HEL) emulsified in complete Freund's adjuvant (CFA). Two weeks later, animals were boosted i.p. with the same amount of HEL in incomplete Freund's adjuvant (IFA). One week after boosting, the animals were bled and the serum was tested for the presence of HEL-specific IgG by ELISA. The y axis represents OD₄₁₅₋₄₉₀×1000. In FIG. 2 B, RAG-(syn) mice (striped bars; mice #61 and 62) and RAG-(allo) mice (speckled bars; mice #46 and 51) were immunized in the hind foot pads with a total of 50 μg of KLH emulsified in CFA on d=0. The animals were boosted subcutaneously two weeks later with KLH in IFA. Serum samples were taken at d=0, 14 and 21 days, and then tested for KLH-specific IgG by ELISA. The plain bars on the graph represent individual control mice: (C57B1/6×129) Fl mice are represented by the open bars, and Balb/c mice by the gray bars. The y axis indicated the OD₄₁₅₋₄₉₀×1000 for a 1:1000 dilution of serum. The x axis represents days post-primary immunization. Specificity was tested by ELISA on HEL coated plates; no cross-reactivity was seen.

FIGS. 3A-3B demonstrate that antigen specific T cell proliferative responses are restricted to both donor and host MHC in neonatally constructed RAG-(allo) chimeras. RAG-(allo) mice #135 (A) and 136 (B) were immunized in the hind foot pad with KLH in CFA. Ten days later, draining lymph nodes were removed and depleted of B cells and macrophages by cytotoxic elimination. The resulting lymph node T (LNT) cells were co-cultured for three days with a 2:1 ratio of Mitomycin C-fixed, antigen-pulsed LPS-blasts from Balb/c and C57B1/6 mice. Proliferative responses were quantitated using a colorometric assay (CellTiter; Promega). Background responses were subtracted. LNT from immunized Balb/c mice gave OD₄₉₀×1000=547 in response to antigen pulsed Balb/c blasts.

FIG. 4 is a schematic diagram setting forth the steps taken to generate a recipient mammal expressing growth factor transgenes.

FIG. 5 demonstrates expression of particular human transgenes in specific tissues of clones 71, 74 and 75, in which expression was determined by reverse transcriptase PCR (RT-PCR). FIG. 5 also shows expression of the corresponding endogenous genes in specific tissues of the mouse for comparison.

FIGS. 6A and 6B present results of analysis of levels of expression of certain transgenes in recipient clones in either serum or bone marrow stromal cells (FIG. 6A), and the sensitivity of the test, as well as the normal range of expression and average expression of the growth factor transgenes in humans (FIG. 6B).

FIGS. 7A-7B are bar graphs showing the level of expression of human M-CSF protein in certain clones of transgenic mice. In FIG. 7B, the numbers within the clones refer to individual mice.

FIG. 8 is a schematic diagram outlining the methodology used for determining whether bone marrow stromal cells obtained from an i-mune mouse expressing human growth factors can support human hematopoiesis in vitro.

FIG. 9 is a graph showing the ability of bone marrow stromal cells derived from i-mune mice of the present invention and control mice to maintain levels of human non-adherent cells in vitro.

FIG. 10 is a graph showing the ability of bone marrow stromal cells derived from i-mune mice of the present invention and control mice to maintain production of human myeloid progenitor cells in vitro.

FIG. 11 is a bar graph showing the ability of bone marrow stromal cells derived from i-mune mice of the present invention and control mice to maintain production of human myeloid progenitor cells in vitro. This graph was generated using the same data used to generate FIG. 10.

FIG. 12 is a bar graph of additional data from a second set of experiments showing the ability of bone marrow stromal cells derived from i-mune mice of the present invention and control mice to maintain production of human myeloid progenitor cells in vitro.

FIG. 13 is a bar graph showing human myeloid progenitor production using bone marrow stromal cells obtained from the i-mune mouse clones and wild-type strains at four weeks.

### 5. DETAILED DESCRIPTION OF THE INVENTION

It has been well established using a variety of model systems that thymic cortex epithelial cells perform the majority of the positive selection events that occur during T cell differentiation (Paul, ed. Fundamental Immunology, 4^{th} Ed. (1999), Lipincott-Raven Press). Recently, attempts to further define the cell types involved in positive selection have revealed a dichotomy in the ability of CD4⁺ and CD8⁺ single positive cells to be selected by bone marrow (BM)-derived cells. It has been conclusively demonstrated that CD8⁺ T cells can be positively selected by hematopoietic cells using chimeric animals constructed on an MHC class I deficient background (Bix & Raulet, Nature 359:330-333 (1992)). However, the opposite result has been shown for CD4⁺ T cells using a similar model system employing irradiated MHC class II deficient mice (Markowitz, et al., Proc. Nat'l Acad. Sci. 90(7):2779-83 (1993)). These results suggest that selection events are more stringently controlled for CD4⁺ than for CD8⁺ T cells.

The present invention is based, in part, on the fact that the inventors have found that fully allogeneic chimeric animals generated either directly after birth, or in adult, non-irradiated antigen receptor recombination-deficient, e.g., recombination activation gene-2 (RAG-2) mutant, mice possess CD4⁺ T cells in the periphery that exhibit donor MHC restricted antigen-specific responses. These results have not been seen in either neonatally or adult constructed SCID chimeras. This suggests that hematopoietic cells are capable of positively selecting CD4⁺ T cells in the thymus, and present antigen receptor recombination-deficient strains of mammals as a unique model system which may support T cell development more closely resembling normal ontogeny. It appears that positive selection of CD4⁺ T cells by hematopoietic cells has not been routinely detected in other systems due to the use of incompletely immunoincompetent mice, and/or due to secondary effects of irradiation.

The present invention is also based, in part, on the fact that the inventors have further discovered methods by which xenogeneic transgenes required for the growth and development of a xenogeneic hematopoietic stem cells are incorporated into the host mammal. After incorporation, cytokine transgenic (CTG) mammals engrafted with xenogeneic hematopoietic stem cells (HSC) develop a functional immune system capable of donor MHC-restricted antigen-specific responses. This modification provides a pathway for donor lymphocyte development in the context of xenogeneic MHC molecules expressed on the MHC-expressing tissues of the host. These mammals can then be used as a model system for human or other mammalian diseases.

5.1 Production of the Mammals of this Invention

In a preferred embodiment, the recipient mammals of this invention are immunodeficient. To produce immunodeficient mammals, the naturally occurring immune systems of the mammals should be inactivated. Inactivation can take place by removing or disrupting multiple immune system-related activities or by removing or disrupting just one activity. Although immune function can be disrupted by many different mechanisms, e.g., spontaneous mutation, irradiation and antisense technology, in a preferred embodiment, immune function is disrupted by knocking out by e.g., homologous recombination or spontaneous mutation, one or more gene functions necessary for maturation and maintenance of the immune system.

5.1.1 Generation of Knock Out Mammals

Homologous recombination may be employed for gene replacement, inactivation or alteration of genes. A number of papers describe the use of homologous recombination in mammalian cells. See, for example, Thomas & Capecchi, Cell 51:503 (1987); Nandi, et al., Proc. Nat'l Acad. Sci. USA 85:3845 (1988); and Mansour, et al., Nature 336:348 (1988); Schweizer, et al., J. Biol. Chem. 274:20450 (1999); Hauser, et al., Proc. Nat'l Acad. Sci. USA 96:8120 (1999); Haber, Trends Biochem. Sci. 24:271 (1999); and Bonaventure, et al., Mol. Pharmacol. 56:54 (1999).

Furthermore, various aspects of using homologous recombination to create specific genetic mutations in embryonic stem cells and to transfer these mutations to the germline have been described (Thomas & Capecchi, Cell 51:503 (1987); Thompson, et al, Cell 56:316 (1989); Antoine, et al., J. Cell Sci. 112:2559 (1999); Molotkov, et al., Cancer Lett. 132:187 (1998); Bleich, et al., Pflugers Arch. 438:245 (1999); Struble, et al., Neurosci. Lett. 267:137 (1999); Schweizer, et al., J. Biol. Chem. 274:20450 (1999); Cuzzocrea, et al., Eur. Cytokine Netw. 10:191 (1999); and Mombaerts, et al. Cell 68:869-77 (1992); and Shinkai, et al. Cell 68:855-867 (1992)).

Thus, the recipient mammals of this invention, which lack necessary endogenous gene(s) necessary for the maturation of lymphocytes, can be made using homologous recombination to effect targeted gene replacement. In this technique, a specific DNA sequence of interest is replaced by an altered DNA. In a preferred embodiment, the genome of an embryonic stem (ES) cell from a desired mammalian species is modified (Capecchi, Science 244:1288 (1989) U.S. Pat. No. 5,487,992).

As mentioned above, the gene to be replaced by homologous recombination is one that is activated early in lymphocyte development. Without being bound by any particular theory, it is believed the desired gene is activated while the thymocyte is in the CD4⁻ and CD8⁻ state (double negative) or the CD44^{low} and CD25⁺ state, and the B lymphocyte is in the B220^{dull}/CD43+ state. Because at these states, T and B cell receptor rearrangement occurs, it is believed the genes that encode proteins that modulate the VDJ recombination are likely targets for replacement. Examples of these genes are the RAG-1 and RAG-2 genes, the T cell receptor (TCR) and immunoglobulin (Ig) genes, the CD3 genes, the pre-T cell receptor, and the SCID gene. Additional types of genes that regulate the survival and differentiation of lymphocyte precursors are also potential targets, e.g., the ikarus transcription factor, the common gamma chain subunit, IL-7, and the IL-7 receptor, among others.

The procedures employed for inactivating one or both copies of a gene coding for a particular protein that modulates early thymocyte development will be similar, differing primarily in the choice of sequence, selectable marker used, and the method used to identify the absence of the modulating protein, although similar methods may be used to ensure the absence of expression of a particular protein. Since the procedures are analogous, the inactivation of the RAG-2 gene in mice will be used as an example. See, U.S. Pat. No. 5,859,307, the entirety of which is incorporated by reference.

The homologous sequence for targeting the construct may have one or more deletions, insertions, substitutions or combinations thereof. For example, the RAG-2 gene may include a deletion at one site and/or an insertion at another site. The presence of an inserted positive marker gene will result in a defective inactive protein product insertion as well as a gene that can be used for selection. Preferably, deletions are employed. For an inserted gene, of particular interest is a gene which provides a marker, e.g., antibiotic resistance such as neomycin resistance, including G418 resistance.

The deletion should be at least about 50 base pairs, or more usually at least about 100 base pairs, and generally not more than about 20,000 base pairs, where the deletion will normally include at least a portion of the coding region including a portion of or one or more exons, a portion of one or more introns, and may or may not include a portion of the flanking non-coding regions, particularly the 5′-non-coding region (transcriptional regulatory region). Thus, the homologous region may extend beyond the coding region into the 5′-non-coding region or alternatively into the 3′-non-coding region. Insertions should generally not exceed 10,000 base pairs, usually not exceed 5,000 base pairs, generally being at least 50 base pairs, more usually at least 200 base pairs.

The homologous sequence should include at least about 100 base pairs, preferably at least about 150 base pairs, and more preferably at least about 300 base pairs of the target sequence and generally not exceeding 20,000 base pairs, usually not exceeding 10,000 base pairs, and preferably less than about a total of 5,000 base pairs, usually having at least about 50 base pairs on opposite sides of the insertion and/or the deletion in order to provide for double crossover recombination.

Upstream and/or downstream from the desired DNA may be a gene which provides for identification of whether a double crossover has occurred. For this purpose, the herpes simplex virus thymidine kinase gene may be employed, since the presence of the thymidine kinase gene may be detected by the use of nucleoside analogs, such as Acyclovir or Gancyclovir, for their cytotoxic effects on cells that contain a functional HSV-tk gene. The absence of sensitivity to these nucleoside analogs indicates the absence of the thymidine kinase gene and, therefore, where homologous recombination has occurred, that a double crossover event has also occurred.

The presence of the marker gene inserted into the RAG-2 gene of interest establishes the integration of the targeting construct into the host genome. However, DNA analysis might be required in order to establish whether homologous or non-homologous recombination occurred. This can be determined by employing probes for the target DNA sequence that hybridize to the 5′ and 3′ regions flanking the insert. The presence of an insert, deletion, or substitution in the targeted gene, can be determined using restriction endonucleases that distinguish the size of a targeted allele from a wild type allele.

The polymerase chain reaction may also be used in detecting the presence of homologous recombination (Kim & Smithies, Nucleic Acid Res. 16:8887-8903 (1988); and Joyner, et al., Nature 338:153-156 (1989)). Primers may be used which are complementary to a sequence within the construct and complementary to a sequence outside the construct and at the target locus. In this way, one can only obtain DNA duplexes having both of the primers present in the complementary chains if homologous recombination has occurred. By demonstrating the presence of the primer sequences or the expected size sequence, the occurrence of homologous recombination is supported.

The construct may further include an origin of replication which is functional in the mammalian host cell. For the most part, these replication systems will involve viral replication systems, such as Simian Virus 40, Epstein-Barr virus, papilloma virus, adenovirus and the like.

Where a marker gene is involved, as an insert, and/or flanking gene, depending upon the nature of the gene, it may have the wild-type transcriptional regulatory regions, particularly the transcriptional initiation regulatory region or a different transcriptional initiation region. Whenever a gene is from a host where the transcriptional initiation region is not recognized by the transcriptional machinery of the mammalian host cell, a different transcriptional initiation region will be required. This region may be constitutive or inducible, preferably inducible. A wide variety of transcriptional initiation regions have been isolated and used with different genes. Of particular interest as promoters are the promoters of metallothionein-I and II from a mammalian host, thymidine kinase, beta-actin, immunoglobulin promoter, human cytomegalovirus promoters, phosphoglycerate kinase (PGK) and SV40 promoters. In addition to the promoter, the wild-type enhancer may be present or an enhancer from a different gene may be joined to the promoter region.

The construct may further include a replication system for prokaryotes, particularly E. coli, for use in preparing the construct, cloning after each manipulation, allowing for analysis, such as restriction mapping or sequencing, followed by expansion of a clone and isolation of the construct for further manipulation. When necessary, a different marker may be employed for detecting bacterial transformants.

Once the construct has been prepared and manipulated and the undesired sequences removed from the vector, e.g., the undesired bacterial sequences, the DNA construct is now ready to be introduced into the target stem cells. Methods of introducing the desired DNA into stem cells are well known in the art. Briefly, preferred methods include, but are not limited to calcium phosphate/DNA coprecipitates, microinjection of DNA into the nucleus, electroporation, bacterial protoplast fusion with intact cells, lipofection, or the like. The DNA may be single or double stranded, linear or circular, relaxed or supercoiled DNA. For various techniques for transforming mammalian cells, see Keown, et al., Methods in Enzymology 185:527-537 (1990); Sambrook, et al., MOLECULAR CLONING: A LABORATORY MANUAL (2ND ED.), VOLS. 1-3, Cold Spring Harbor Laboratory, (1989) (“Sambrook”) or CURRENT PROTOCOLS IN MOLECULAR BIOLOGY, F. Ausubel et al., ed. Greene Publishing and Wiley-Interscience, New York (1987) (“Ausubel”).

After transformation of the target cells, many target cells are selected by means of positive and/or negative markers, as previously indicated, neomycin resistance and Acyclovir or Gancyclovir resistance. Those cells which show the desired phenotype may then be further analyzed by restriction analysis, electrophoresis, Southern analysis, polymerase chain reaction or the like. By identifying fragments which show the presence of the mutations at the target gene site, one can identify cells in which homologous recombination has occurred to inactivate the target gene.

Cells in which only one copy of the, e.g., RAG-2, gene have been inactivated still retain a single unmutated copy of the target gene. If desired, these cells can be expanded and subjected to a second transformation with a vector containing the desired DNA. If desired, the mutation within the desired DNA may be the same or different from the first mutation. If a deletion, or replacement mutation is involved, a second mutation may overlap at least a portion of the mutation originally introduced. If desired, a different positive selection marker can be used in this transformation. If a different marker is used, cells with both mutations can be selected in double selection media. Alternatively, to determine if the cells comprise mutations in both copies of the transformed cells, the cells can be screened for the complete absence of the functional protein of interest. The DNA of the cell may then be further screened to ensure the absence of a wild-type target gene.

In an alternative embodiment, chimeric mammals can be developed from transformed stem cells (see, infra) and animals with one mutated sequence can be bred to other mammals with one or two mutated sequences and offspring that contain mutations in both copies (homozygotes) selected as recipient mammals of this invention. Similarly, recipient mammals developed from chimeric mammals from transformed cells with two mutated genes can be bred to produce more recipient mammals.

After transformation, the stem cells containing either one or two copies of the replacement DNA are inserted into recipient mammal embryos to produce chimeric mammals. Typically, this is done by injecting stem cell clones into mammalian blastocysts. The blastocysts are then implanted into pseudopregnant females. The offspring derived from the implanted blastocysts are test-mated to animals of the parental line to determine whether the offspring comprise a chimeric germ line. Chimeras with germ cells derived from the altered stem cells transmit the modified genome to the offspring of the test matings, yielding mammals heterozygous for the target DNA (contain one target DNA and one replacement DNA). The heterozygotes are then bred with each other to create homozygotes for replacement DNA.

Because the recipient mammals of this invention are immunodeficient, it may be necessary to maintain them in a germ free environment. Such environments are well known to those of skill in the art and techniques for maintaining immunodeficient mice can be found in Immunodeficient rodents: a guide to their immunobiology, husbandry, and use, Committee on Immunologically Compromised Rodents, Institute of Laboratory Animal Resources, Commission on Life Sciences, National Research Council. Washington, D.C.: National Academy Press, 1989.

In addition to producing knock-out mammals, the immunodeficient mammals of this invention are commercially available. For example, mice with a RAG-2 mutation are available from Taconic, RAG-1 and TCRbeta/delta mutant mice from Jackson Laboratory, or SCID mice from Jackson and Taconic.

In another embodiment, introduction of transcriptionally active transgenes, e.g., a truncated forms of rearranged antigen receptors or human CD3 epsilon, are examples of achieving lymphocyte deficiencies.

It is desireable to screen the recipient mammals for the presence of the knocked out gene. Screening can be done phenotypically or genotypically. Phenotypic screening includes, but is not limited to, the absence of mature T and B cells and other phenotypic changes that correlate with the absence of mature T and B cells, such as the absence of serum immunoglobulins. However, if the mutated gene presents as a dominant phenotype, animals that are heterozygous at that gene will present with the same phenotypic characteristics as the desired homozygotes. Therefore, it is desirable to screen for homozygotes by genotypic screening.

DNA screening is well known to those of skill in the art and can be found in, for example, Ausubel and Sambrook. Briefly, cells containing DNA are removed from the test animals. In mice, this can be done by removing the tip of the tail and isolating cells. The genomic DNA is isolated from the cells and cut into manageable size by restriction endonucleases. The cut genomic DNA is electrophoresed in an agarose gel and then probed with a labeled nucleic acid that can distinguish the wild type from the modified DNA fragment.

Binding of the labeled probe to the genomic DNA depends on the ability of the probe to remain hybridized to the genomic DNA under the wash conditions used. An extensive guide to the hybridization of nucleic acids is found in Tijssen, Laboratory Techniques in Biochemistry and Molecular Biology—hybridization with Nucleic Acid Probes, Elsevier, New York (1993). Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or northern blot is 50% formalin with 1 mg of heparin at between 40 and 50° C., preferably 42° C., with the hybridization being carried out overnight. An example of highly stringent wash conditions is 0.15M NaCl at from 70 to 80° C. with 72° C. being preferable for about 15 minutes. An example of stringent wash conditions is 0.2×SSC wash at about 60 to 70° C., preferably 65° C. for 15 minutes (see, Sambrook, supra for a description of SSC buffer). Often, a high stringency wash is preceded by a low stringency wash to remove background probe signal. An example medium stringency wash for a duplex of, e.g., more than 100 nucleotides, is 1×SSC at 40 to 50° C., preferably 45° C. for 15 minutes. An example low stringency wash for a duplex of, e.g., more than 100 nucleotides, is 4-6×SSC at 35 to 45° C., with 40° C. being preferable, for 15 minutes. In general, a signal to noise ratio of 2× (or higher) than that observed for an unrelated probe in the particular hybridization assay indicates detection of a specific hybridization. After removal of unbound probe, the label is detected and the presence or absence of the desired DNA in the genome of the mammals determined.

As the mammal matures, it may exhibit a “leaky phenotype.” For purposes of this invention, a leaky phenotype is one where a few thymocytes and/or pro-B cells undergo functional receptor rearrangement and mature into T and B cells, respectively. Thus, a SCID mouse exhibits a leaky phenotype. This phenotype can be detected by monitoring the development of host T and B cells and/or serum immunoglobulin in the recipient mammals throughout the life of the animal.

5.1.2 Transgenic Mammals

The differentiation of hematopoietic cells is a highly regulated process that involves the coordinate expression of many factors, including cytokines, adhesion molecules, and chemokines, among others. Due to evolutionary changes, considerable divergence has occurred between a number of murine and human growth factors such that the murine factors do not always interact as efficiently, or in the same manner, as their human counterparts. A major consideration when supplying exogenous cytokines is the dosage, combination, and pattern of delivery. Since cytokines are powerful signaling molecules that work in close proximity to their origin, in low concentrations, and synergistically with one another, systemic delivery of exogenous cytokines is unlikely to provide the physiological levels necessary for normal development.

The preferred method of providing human-specific factors to the host is via transgenesis, whereby copies of genomic DNA encoding the desired factors are incorporated into the genome of the host. The DNA should include tissue-specific regulatory sequences and any introns and exons required for normal RNA processing, including alternatively spiced variants. The latter may be particularly important when the proteins present themselves as both membrane bound and soluble forms having different physiological effects. Thus, to maintain a donor species-specific functional immune system, it is necessary to introduce donor-specific cytokines into the germline of the recipient mammals of this invention.

The recipient mammals of this invention are produced by introducing transgenes into the germline of a non-human animal. Embryonal target cells at various developmental stages can be used to introduce transgenes. Different methods are used depending on the stage of development of the embryonal target cell. For example, the zygote is the best target for micro-injection. In the mouse, the male pronucleus of the zygote reaches approximately 20 micrometers in diameter. At this size, reproducible injections of 1-2 pL of DNA solution can be performed. The use of zygotes as a target for gene transfer has another major advantage in that, in most cases, the injected DNA will be incorporated into the host genome before the first cleavage (Brinster, et al. Proc. Natl. Acad. Sci. USA 82:4438-4442 (1985)). As a consequence, all cells of the recipient mammal will carry the incorporated transgene. This is also reflected in the efficient transmission of the transgene to offspring of the parent transgenic mammal since 50% of the germ cells of the offspring will harbor the transgene.

In another, alternative embodiment, intracytoplasmic sperm injection (ICSI) can be used to introduce transgenes into metaphase oocytes. See, Perry, et al., Science 284:1180 (1999). Briefly, sperm heads and linearized DNA are incubated for a short period of time and co-injected into an oocyte. Improved rates of transgenesis are seen when the sperm heads have undergone membrane disruption prior to incubation with the DNA.

Retroviral infection can also be used to introduce a transgene into a recipient mammal. The developing embryo can be cultured in vitro to the blastocyst stage. The blastomeres are then targets for retroviral infection (Jaenisch, Proc. Na''l Acad. Sci USA 73:1260-1264 (1976)). Efficient infection of the blastomeres is obtained by enzymatic treatment to remove the zona pellucida (Hogan, et al., MANIPULATING THE MOUSE EMBRYO, Cold Spring Harbor Laboratory Press, Cold Spring Harbor, N.Y. (1986)). The viral vector system used to introduce the transgene is typically a replication-defective retrovirus carrying the transgene (Jahner, et al. Proc. Natl. Acad. Sci. USA 82: 6927-6931 (1985); Van der Putten, et al. Proc. Natl. Acad. Sci USA 82: 6148-6152 (1985)). Infection is easily and efficiently obtained by culturing the blastomeres on a monolayer of virus-producing cells (Van der Putten, supra; Stewart, et al. EMBO J. 6: 383-388 (1987)). Alternatively, infection can be performed at a later stage. Virus or virus-producing cells can be injected into the blastocoel (Jahner, D., et al. Nature 298:623-628 (1982)). Most of the founders will be mosaic for the transgene since incorporation occurs only in a subset of the cells which form the recipient mammal. Further, the founder may contain various retroviral insertions of the transgene at different positions in the genome which generally will segregate in the offspring. In addition, it is also possible to introduce transgenes into the germ line, albeit with low efficiency, by intrauterine retroviral infection of the midgestation embryo (Jahner, D. et al. supra).

A fourth type of target cell for transgene introduction is the embryonal stem cell (ES). ES cells are obtained from pre-implantation embryos cultured in vitro and fused with embryos (Evans, et al. Nature 292:154-156 (1981); Bradley, et al. Nature 309:255-258 (1984); Gossler, et al. Proc. Natl. Acad. Sci USA 83:9065-9069 (1986); and Robertson, et al. Nature 322:445-448 (1986)). Transgenes can be efficiently introduced into the ES cells by DNA transfection or by retrovirus-mediated transduction. Such transformed ES cells can thereafter be combined with blastocysts from a nonhuman animal. The ES cells thereafter colonize the embryo and contribute to the germ line of the resulting chimeric recipient mammal. For review see Jaenisch, Science 240:1468-1474 (1988); Bradley, et al. Biotechnology (N Y) 10(5):534-9 (1992); and Williams, Bone Marrow Transplant 5(3):141-4 (1990).

The actual transgenes of this invention include the coding sequences for proteins necessary for the maturation and maintenance of a donor-specific functional immune system. Those of skill will recognize the required cytokines will vary depending on the desired functionality. Such cytokines include but are not limited to, IL-6, IL-7, GM-CSF and SCF, LIF, M-CSF, and OM. In addition or alternatively, MHC genes from the same species and haplotype as that of the donor HSC may be introduced into the recipient mammal and expressed in tissues that endogenously express MHC molecules. In this case, donor thymocytes become “restricted” during development in the recipient's tissues, particularly the thymus, via interaction with the transgenic MHC molecules. This leads to the maturation of T cells that can have cognate interactions with donor B lymphocytes displaying the same haplotype of transgenic MHC molecules as the host mammal. For example, human HLA -DR, -DQ, and/or -DP genes of the same haplotype as the HSC donor are expressed in the mouse tissues. The expression of transgenic MHC expression is most beneficial in mouse strains other than those with mutations in the RAG-2 or RAG-1 genes.

In a preferred embodiment, the cytokine genes are derived from a human being or a human cell line. However, other mammalian sources may be used such as pig, sheep or rat. In an alternative embodiment, mammals of the same species but allogeneic to the donor are the source of the cytokines.

There are numerous other methods of isolating the DNA sequences encoding the cytokines of this invention. For example, DNA may be isolated from a genomic or cDNA library using labeled oligonucleotide probes having sequences complementary to known cytokine sequences. For example, full-length cDNA probes may be used, or oligonucleotide probes consisting of subsequences of the known sequences may be used. Such probes can be used directly in hybridization assays to isolate DNA encoding cytokines. Alternatively probes can be designed for use in amplification techniques such as PCR, and DNA encoding cytokines may be isolated by using methods such as PCR.

To prepare a cDNA library, mRNA is isolated from a source tissue or cells. For example, I1-7 is expressed by bone marrow stroma, thus, these cells would be a suitable source of mRNA that encodes I1-7. cDNA is reverse transcribed from the mRNA according to procedures well known in the art and inserted into bacterial cloning vectors. The vectors are transformed into a recombinant host for propagation, screening and cloning. Methods for making and screening cDNA libraries are well known. See, Gubler & Hoffman, Gene 25:263-269, (1983) and Sambrook, et al.

From a genomic library, total DNA is extracted from the host tissue or cells and then cut into smaller pieces of DNA by mechanically shearing or by enzymatic digesting to yield fragments of about e.g., 12-50 kb. Fragments of a desired size are then separated by gradient centrifugation and are inserted into bacteriophage lambda vectors or other vectors. These vectors and phage are packaged in vitro, as described in Sambrook, et al. Recombinant phage can be analyzed for the presence of cytokine nucleic acids by plaque hybridization as described in Ausubel.

Libraries containing genomic DNA sequences greater than 50 kb are prepared using various cloning vectors, e.g., YAC, BAC, P1, and PAC vectors. Techniques for generating these libraries are well known. See, Markie, ed. (for YACS) Methods in Molecular Biology 54 (1997), Ramsay (for YACS), Mol Biotechnol 1(2):181-201 (1994), Monaco et al., Trends Biotechnol. 12:280-6 (1994), and Shepherd et al., Genet Eng (NY) 16:213-28 (1994).

Hybridization probes useful in this invention include known sequences that encode for the cytokines of interest or sequences that encode homologous cytokines from another species, for example a probe derived from a murine sequence to probe a human cDNA library for a homologous sequence. One of skill will recognize that if homologous sequences are used as probes, the stringency of the wash conditions should be lowered.

In addition to generating coding sequences of the cytokines to be used as transgenes, many of the nucleic acids that encode the necessary cytokines of this invention are commercially available. Such resources include R&D Systems, Genetic Systems, and CEPH.

The preferred method of making transgenic mammals that express the necessary cytokines is as follows. From both animal and in vitro studies, IL-3, IL-6, IL-7, M-CSF, GM-CSF, stem cell factor, LIF and oncostatin M appear to either play a role in hematopoiesis, are expressed in the bone marrow or thymus, or the murine proteins show specificity for murine vs. human cells, thus suggesting that human HSC engrafted in a recipient mammal may not recognize the native mammalian cytokine. Genomic clones for these human genes can be obtained and transfected into ES cells. The ES cells can be introduced into blastocysts to transfer the donor transgenes into the germline of a recipient mammal.

5.1.3 Isolation of Genomic Clones

In a preferred method, PCR primer sets are designed against either the 5′ or the 3′ end of genomic sequences so that constructs containing the genes can be identified by PCR. In addition, these primer sets can be designed to distinguish between mouse and human genes so that the native mammalian genes are not mistakenly identified during a genomic or transcriptional screen of the transfected ES cells or suspected recipient mammals.

Human genomic libraries are screened using gene specific primer sets (See Example 2 and Ausubel for a general description of genomic library screening). If desired, positive clones can be further confirmed by either other primer sets for the same gene or by Southern blot analysis. Depending on the size of the gene, different types of cloning vectors and libraries are readily available. Genes up to approximately 15 kb may be obtained from lambda libraries. Those up to 50 kb may be identified from cosmid libraries. Larger genes over 50 kb can be isolated from BAC, PAC, P1, YAC, MAC, or other such libraries.

5.1.4 Demonstration of In Vitro Transcription from the Genomic Constructs

Because the transgenes will be expressed in mammalian hosts, it may be desirable to determine the ability of the human sequences to be transcribed by mammalian cells other than human, preferably murine before transfection into possibly rare ES cells. Undigested or digested genomic constructs can be transfected by lipofection into a murine cell line that expresses the endogenous form of each cytokine. Commercial lipofection reagents are widely available and may require optimization for a particular cell type to obtain adequate transfection efficiencies in a transient assay. The mRNA from the transfected cells is then analyzed for transcription of the contruct. Depending on the preference of one of skill, the mRNA can be electrophoresed according to standard procedures and then probed in a northern blot, or first strand cDNA can be synthesized by standard reverse transcription methods. The resulting cDNA can then be analyzed by labeled probe and Southern blot or by PCR methods.

5.1.5 Selection of ES Clones that Contain Human Cytokine Genes

Two equally preferred embodiments can be used to combine all of the desired genes into one strain. In the first method, groups of transgenes are co-transfected into ES cells along with a selectable marker for neomycin resistance. For example, one group of genes can contain IL-7, SCF, and LIF constructs, and the second contain GM-CSF, M-CSF, and IL6 constructs. In the second method, all desired genes are co-transfected together. If all of the necessary transgenes are not present in the germline of one transgenic mammal, that mammal can be mated to a mammal comprising, in its germline, the necessary transgene to create offspring with all necessary transgenes.

To introduce the transgenes into the ES cells, the DNA constructs are digested with a desired restriction endonuclease that linearizes the DNA, if circular. Within each group, it is preferred that DNA constructs are mixed in equal molar ratio. However, one of skill will recognize that if more copies of one gene is desired, DNA constructs of that gene should be over-represented. For positive selection, a marker-containing plasmid can be mixed with these DNAs at molar ratio of about 4:1.

The DNA can then be introduced ES cells by lipofection or another suitable technique. The preferred transfection protocol is similar to that provided by the manufacturer of the lipofection reagent and is described in detail in Example 2. After a suitable time in selection media (preferably 5-20 days and more preferably 10-14 days), individual transfected ES cell colonies are transferred into 96-well dishes for cloning and expansion.

Although one method is described here and in Example 2, those of skill in the art will realize that other methods of inserting transgenes in the germ line of mammals are known and also available. Some of these methods can are found in U.S. Pat. Nos. 4,873,191, 5,434,340, 4,464,764, 5,487,992, 5,814,318; PCT published patent applications WO 97/20043, WO 99/07829, WO 99/08511; and Perry, et al., Science 284:1180 (1999).

Depending on the transgene that has been inserted into the ES cell, different techniques can be used to detect the transgene. For example, PCR can be used to detect genomic DNA or cDNA made from RNA transcripts, ELISA and other antibody-based assays can be used to determine whether the gene product of the transgenes are synthesized in ES cells or are present extracellularly, and if such an assay is available, a functional assay can be used to detect the gene product.

5.1.6 Hematopoietic Stem Cells

Sources of hematopoietic stem cells include, but are not limited to, umbilical cord blood (CB), bone marrow (BM) and mobilized peripheral blood (MPB).

Human CB can be obtained, for example, from Advanced Bioscience Resources Inc (ABR), Alameda, Calif., or Purecell, San Mateo, Calif. CB is collected by ABR from local hospitals within 24 hours of shipping and is processed on site. Alternatively, human BM, CB, and/or MPB cells are obtained from Purecell as either fresh or frozen cells, and fractionated or unfractionated cells. Before use, all samples are tested for Hepatitis B and C, and HIV. Any experimental materials involving samples found to be virus positive are discarded immediately and animals removed, marked and disposed of in accordance with procedures for disposing contaminated animal carcasses. Throughout the course of the experiment, all samples should be treated under the assumption that they may be contaminated with human blood borne pathogens (Biosafety Level 2, BL-2). All personnel handling mice with human blood cells should receive the hepatitis B vaccine.

5.2 Uses of the Mammals of the Invention

The mammals of the present invention can be used to derived long term bone marrow cultures useful for studying hematopoiesis in vitro. Further, the long term bone marrow cultures can be used to maintain donor animal-specific hematopoietic cells in vitro.

In still another use of the invention, factors involved in regulation of the development and function of hematopoietic cells can be determined. These factors can serve to both identify the biological properties of these factors and to test their effectiveness as therapeutic molecules in preclinical models. Of particular interest are factors that augment hematopoietic reconstitution.

### 6. EXAMPLES

The following examples are submitted for illustrative purposes only and should not be interpreted as limiting the invention in any way.

6.1 Allogeneic Reconstitution of RAG-2 Mutant Mice

To compare the ability of CB17.SCID (SCID; H-2^{d}) and RAG2^{-/-} mutant (RAG; H-2^{b}) mice to support development of bone marrow (BM)-derived lymphocyte precursors, animals were engrafted intravenously with 107 T-cell depleted BM cells approximately 72 hours after birth. RAG mice were engrafted with syngeneic (H-2^{b}) C57B1/6 or (129xC57B1/6)F1 BM (RAG-(syn); H-2^{b} [symbol] H-2^{b}) or with fully allogeneic Balb/c BM (RAG-(allo); H-2^{d} [symbol] H-2^{b}). SCID mice were engrafted with syngeneic Balb/c BM (SCID-(syn); H-2^{d} [symbol] H-2^{d}) or with fully allogeneic C57B1/6 BM (SCID-(allo); H-2^{b} [symbol] H-2^{d}).

Hind leg bones (femur and tibia) were taken from euthanized 4 to 8 week old healthy donor mice and flushed using a 25 gauge needle attached to a 3 ml syringe filled with cold DPBS to obtain cells in a single cell suspension. Cells were washed with DPBS and pelleted. Since bone marrow preparations contain functionally mature T cells, T cells were depleted using 10 μg/ml anti-Thy-1 mAb (30H12) at 2-5×10⁷ cells/ml for 30 minutes on ice. Cells were then centrifuged, resuspended in anti-rat IgG (MAR 18.5) culture supernatant at approximately 2-5×10⁷ cells/ml. Guinea pig and rabbit complement were then added to 1:20 v:v each and incubated at 37° for 45 minutes. Cells were centrifuged and resuspended in 3 ml room temperature DPBS, and underlayed with 3 ml room temperature Histopaque density=1.119 and centrifuged for 10 minutes at 800 g. Viable cells were collected from the interface, washed in 2% FCS/DPBS, counted and resuspended in DPBS to a concentration of 3×10⁸ cells/ml with no FCS

Mice were anesthetized using an injectable ketamine/xylazine (100 and 20 mg/kg, respectively) (˜200 μl for average mouse) solution. 3×10⁷ (100 μl) T-depleted bone marrow cells were injected intravenously via the retro-orbital sinus into the appropriate donor (same sex, 5 to 6 weeks of age). Neonatal mice received no more than 50 μl of injectate containing 10⁷ bone marrow cells via the retro-orbital vein with no injectable anesthetic, just reduction of body temperature with ice. Both syngeneic (same MHC haplotype) and allogeneic (MHC haplotype mismatched) mice were transplanted. Mice were left for 8 weeks to allow bone marrow to engraft at which time the mice (usually 3 in each group) were euthanized and organs removed for study. A thorough cellular analysis (FACS) of the thymus, spleen, mesentaeric lymph nodes (in some cases) and peripheral blood (PB) was performed. T cells, B cells, and granulocytes were assessed in these areas using fluorescence-conjugated cell type-specific antibodies. See Table 1.

TABLE 1
Cell numbers and percentages for spleen, lymph node and thymus for
representative 7 to 8 week old neonatally engrafted animals.

| | Spleen | Lymph Nodes | |
|---|---|---|---|
| | Cell # | Cell # | Thymus |
| | (% CD3/B220) | (% CD3/B220) | Cell # (CD4/CD8) |
| SCID (syn) | 5.4 × 10⁷ | 7.8 × 10⁷ | 1.0 × 10⁸ (6/15) |
| | (28/53) | (79/17) | |
| SCID (allo) | 7.5 × 10⁷ | 4.8 × 10⁷ | 2.6 × 10⁸ (3/10) |
| | (30/58) | (65/31) | |
| RAG (syn) | 6.8 × 10⁷ | 2.2 × 10⁷ | 1.0 × 10⁸ (4/10) |
| | (28/33) | (78/18) | |
| RAG (allo) | 3.6 × 10⁷ | 2.6 × 10⁷ | 4.6 × 10⁷ (5.15) |
| | (49/44) | (83/15) | |
| (129xB6)^{a} | 9.9 × 10⁷ | 2.7 × 10⁷ | 11 × 10⁸ (3/10) |
| | (32/58) | (63/29) | |
| Balb/c^{a} | 1.1 × 10⁸ | 2.2 × 10⁷ | 6.6 × 10⁷ (2/12) |
| | (35/54) | (66/23) | |
| RAG 2^{-/-a} | 5.8 × 10⁶ | 3.1 × 10⁵ | 2.5 × 10⁶ (0/0) |
| | (0/5) | (0/3) | |

^{a}averaged data

As can be seen, SCID animals tended to engraft higher percentages of B cells than RAG animals. This was found to be true over a number of different time points. Thymuses of eight week old animals engrafted well, and exhibited normal percentages of CD4⁺ and CD8⁺ single positive cells. Additionally, thymus flow cytometric profiles are comparable, including the percentage of CD3⁺ cells. The RAG (allo) chimera depicted had slightly more immature double negative (DN) thymocytes; however, an enrichment for DN thymocytes was not a reproducible finding over the X number of RAG-(allo) mice examined during the course of this study.

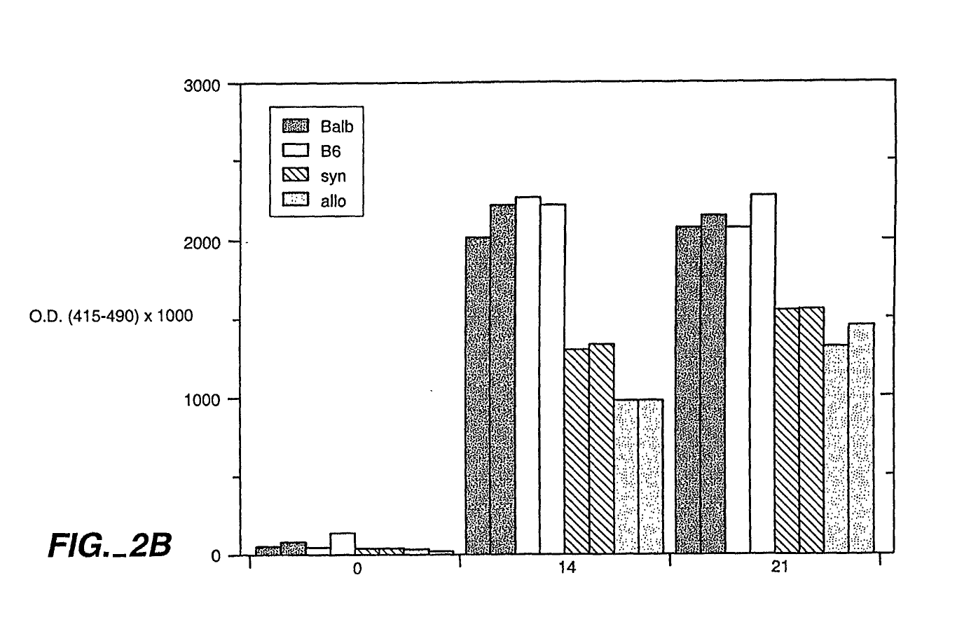

Remaining mice of successful chimera studies were then immunized with 50 μg KLH in CFA intraperitoneally and boosted 2 weeks later with KLH in IFA. Immunized mice were bled 1 week later and sera samples were tested for total IgG as well as IgGl (in some cases) by ELISA. See FIGS. 2A-2B and Table 2 below.

TABLE 2
Characterization of the functional development of
hematopoietic cells in novel allogeneic chimaeras

| Donor strain | Recipient strain | Engraftmt | Ab prod |
|---|---|---|---|
| B6 (H-2^{b}) | 129 RAG-2 | Yes | Yes |
| | (H-2^{b}) | | |
| Balb/c (H-2^{d}) | 129 RAG-2 | Yes | Yes |
| | (H-2^{b}) | | |
| Balb/c (H-2^{d}) | Balb SCID | Yes | N/A |
| | (H-2^{d}) | | |
| B6 (H-2^{b}) | Balb SCID | No | N/A |
| | (H-2^{d}) | | |
| B6 (H-2^{b}) | C1D/129 | CD4's | Yes |
| | RAG-2 (H-2^{b}) | | |
| Balb/c (H-2^{d}) | C1D/129 | CD4s, a few | Slight |
| | RAG-2 (H-2^{b}) | | |
| B6 (H-2^{b}) | C2D/129 | Mediocre | N/A |
| | RAG-2 (H-2^{b}) | | |
| Balb/c (H-2^{d}) | C2D/129 | No | N/A |
| | RAG-2 (H-2^{b}) | | |
| B6 (H-2^{b)} | Irrad C1D/129 | Yes | Yes |
| | RAG-2 (H-2^{b}) | | |
| Balb/c (H-2^{d}) | Irrad C1D/129 | Yes | Yes |
| | RAG-2 (H-2^{b}) | | |
| B6 (H-2^{b}) | Irrad C2D/129 | Yes | Yes |
| | RAG-2 (H-2^{b}) | | |
| Balb/c (H-2^{d}) | Irrad C2D/129 | Yes | Yes |
| | RAG-2 (H-2^{b}) | | |
| Balb/c (H-2^{d}) | Balb RAG-2 | Yes | Yes |
| | (H-2^{d}) | | |
| B6 (H-2^{b}) | Balb RAG-2 | Yes | Yes |
| | (H-2^{d}) | | |
| B6 (H-2^{b}) | B6 SCID | Yes | N/A |
| | (H-2^{b}) | | |
| Balb/c (H-2^{d}) | B6 SCID | No | N/A |
| | (H-2^{b}) | | |
| B6 (H-2^{b}) | (129XB6) | Yes | N/A |
| | RAG-1 (H-2^{b}) | | |
| Balb/c (H-2^{d}) | (129XB6) | No | N/A |
| | RAG-1 (H-2^{b}) | | |
| B6 (H-2^{b}) repeat | (129XB6) | Yes | Yes |
| | RAG-1 (H-2^{b}) | | |
| Balb/c (H-2^{d}) repeat | (129XB6) | No | N/A |
| | RAG-1 (H-2^{b}) | | |
| Balb/c (H-2^{b}) | (129XB6) | No | N/A |
| | RAG-1 (H-2^{b}) 5E7 | | |
| Balb/c (H-2^{b}) | (129XB6) | Yes | Yes |
| | RAG-1 (H-2^{b}) irrad | | |
| Balb/c (H-2^{d}) | 129 RAG-2 | Yes | Yes |
| | (H-2^{b}) | | |
| AKR (H-2^{k}) | 129 RAG-2 | Yes | Yes |
| | (H-2^{b}) | | |
| AKR (H-2^{k}) | 129 RAG-2 | Yes | Unknown |
| | (H-2^{b}) | | |
| TCRtgxAKR (H-2^{k/s}) | 129 RAG-2 | Yes | Unknown |
| | (H-2^{b}) | | |
| B6 (H-2^{b}) | TCR/ko | Yes | Yes |
| | (H-2^{b}) (irrad) | | |
| Balb/c (H-2^{d}) | TCR/ko | Yes | Yes |
| | (H-2^{b}) (irrad) | | |

As has been reported, SCID-(allo) mice responded poorly, indicating a lack of antigen-specific cognate T-B interactions. This has been presumed to be due to positive selection of donor CD4⁺ T cells by the host thymic epithelium, resulting in T cells unable to interact with donor MHC-expressing B cells. In contrast, RAG-(allo) mice produced equivalent levels of antigen-specific IgG as RAG-(syn) animals (FIG. 2A). Pre-bleed serum routinely showed absorbances equal to background. In addition, serum IgG was not cross-reactive when tested on ovalbumin (OVA) coated plates. Antigen specific IgM responses were also noted. This phenomenon was investigated for a second antigen, KLH. As is shown in FIG. 2B for two independent RAG-(allo) animals, the antigen-specific serum IgG response was found to be on the order of control RAG-(syn) responses, and developed with similar kinetics. Serum from all mice was found not to cross-react on ELISA plates coated with an irrelevant antigen. This result suggests that donor derived BM cells are capable of positively selecting CD4⁺ T cells which can then interact with donor derived antigen-specific B cells in the periphery, resulting in an isotype switch to IgG.

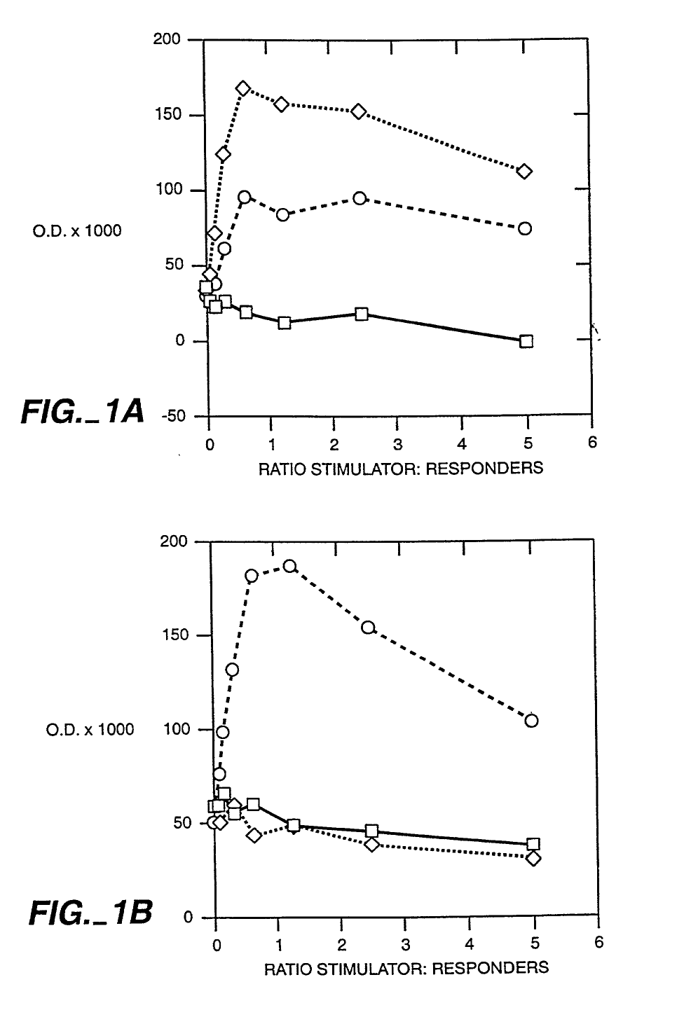

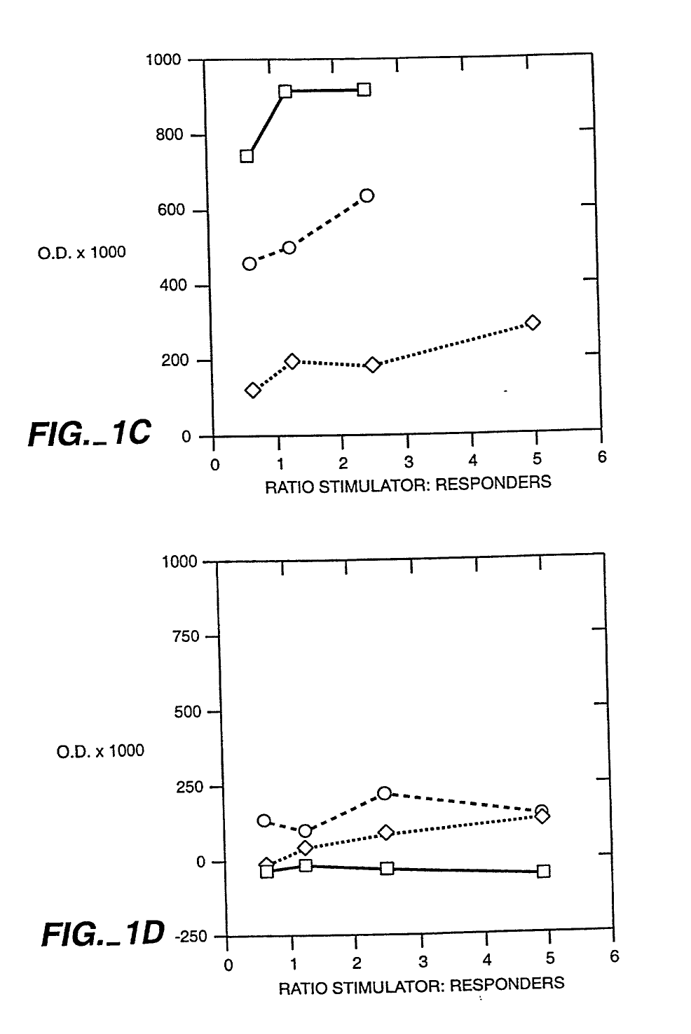

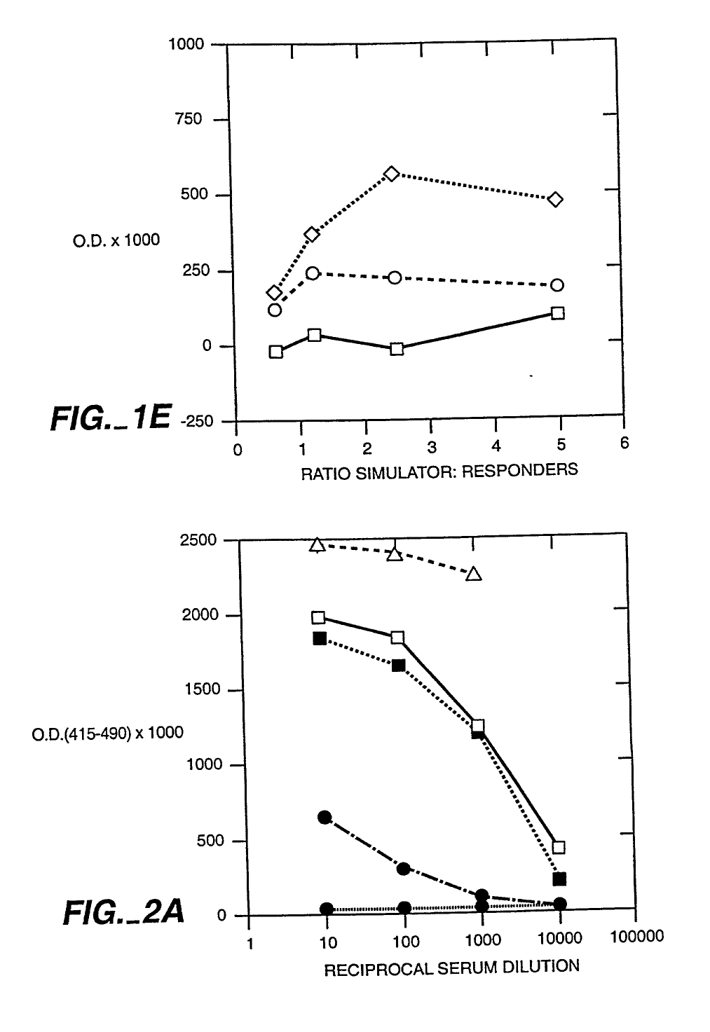

Functional analyses on peripheral lymphocytes from chimeric animals were also performed. CD4⁺ T cells were isolated from the lymph nodes of engrafted animals, and tested for reactivity in a mixed lymphocyte culture (MLR). FIGS. 1A and 1C depict proliferative responses of RAG-(syn) (A) and SCID-(syn) (C) CD4⁺ T cells to LPS-induced splenic blasts from C57B1/6, Balb/c and third party H-2^{k} expressing mice. Both RAG-(syn) and SCID-(syn) were tolerant to self, but were responsive to alloantigens. RAG-(allo) mice were tolerant to both C57B1/6 and Balb/c and were responsive to third party H-2^{k} alloantigens. However, SCID-(allo) was functionally compromised in that a small response was mounted to Balb/c derived stimulators, indicating incomplete tolerance to self MHC. Additionally, the response to third party H-2^{k} expressing stimulators was impaired. A control RAG-(syn) created with the same BM inoculum as injected into the SCID-(allo) is shown in FIG. 1D. Thus, RAG-(allo) mice were found to be tolerant to both donor and host MHC, and were responsive to third party, but SCID-(allo) was functionally impaired. In addition, the mitogen reactivity of splenocytes and lymph node cells was tested. RAG-(allo) splenocytes responded normally to the T cell mitogen PHA, while SCID-(allo) T cells were hyporesponsive. All engrafted animals' splenocytes showed control level responses to LPS.

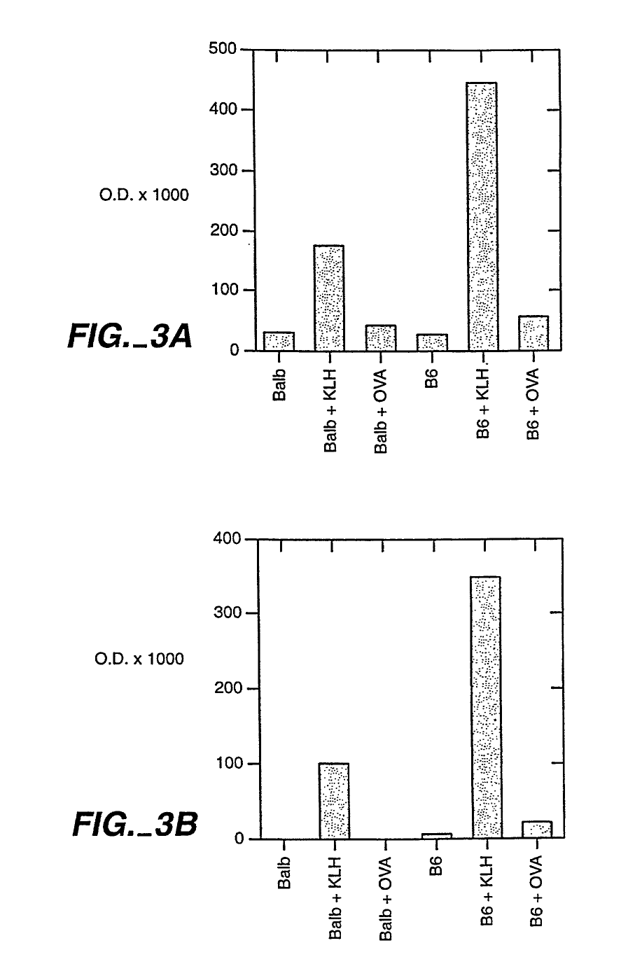

To further investigate the apparent donor restricted T cell responses, RAG-(allo) animals were immunized in the hind foot pads, then CD4⁺ T cells were purified from draining lymph nodes. Primed CD4⁺ T cells were co-cultured with antigen-pulsed LPS-induced splenic blasts, and proliferation was assessed. FIGS. 3A-3B show the proliferative responses of KLH-primed draining CD4⁺ T cells from two different RAG-(allo) animals. The response to KLH pulsed C57B/1/6 stimulators was found to dominate, indicating preferential selection of CD4⁺ T cells to recognize antigen in the context of the thymic epithelium MHC. However, an antigen-specific response to KLH-pulsed Balb/c blasts represented approximately 30% of the control response. This experiment was repeated 5 times, with a similar level of donor restricted response noted (range=20 to 50% of host response). Similar results were obtained with adult engrafted mice.

These results suggest that for the fully allogeneic H-2^{d} [symbol] H-2^{b} combination created in unirradiated neonatal RAG hosts, donor derived BM cells positively selected CD4⁺ T cells. It had been well accepted that thymic epithelium affected the majority of the positive selection occurring in the thymus. However, selection by other cell types and across MHC barriers had been observed only for CD8⁺ T cells. Positive selection of both MHC class I restricted (Pawlowsky, et al. Nature 364:642-5 (1993)) and class II restricted (Hugo, et al. Proc. Natl Acad. Sci. 90:10335-10339 (1993)) T cells had been demonstrated to occur on transfected fibroblasts injected intrathymically. Selection of MHC class II restricted cells was demonstrated when the thymocytes shared MHC haplotypes with both the thymic epithelium and the injected fibroblasts. This constraint was not evident for the selection of MHC class I restricted cells. While thymic positive selection by BM cells for CD4⁺ T cells did not occur in BM engrafted irradiated adult MHC class II-deficient mice, others have demonstrated functional restriction to donor MHC using parental into F1 bone marrow chimeras. On the other hand, functional restriction of CD4⁺ T cells to donor MHC, indicating positive selection by BM-derived cells, has not been demonstrated in fully allogeneic chimeras, although with a few exceptions (Longo, et al. Nature 287:44-47 (1980); Longo, et al. J. Immunol 130:2525-2528 (1983); and Longo, et al. Proc. Nat'l Acad. Sci. 82:5900-5904 (1985)). Taken together these data indicate that thymic epithelium and BM-derived cells must share MHC haplotypes to effect efficient positive selection (Zink, and Elliot), and the requirements for selection of CD4⁺ T cells may be more strict than those for CD8⁺ T cells.

The results presented here suggest that the RAG^{2-/-} mutant strain is unique because it provides an environment that allows for BM derived cell selection events to occur efficiently and in the absence of haplotype sharing by the thymic epithelium. The uniqueness of the RAG mouse may be due to the non-leaky nature of the mutation. The SCID mouse is well known to occasionally develop cells with functional antigen receptors. The development of even a few antigen-receptor positive cells may be enough of a signal to the thymic microenvironment to induce functional changes which preclude the recruitment and/or functionality of donor BM-derived cells capable of positive selection. Both neonatal and adult SCID mice were used in these experiments and neither exhibited the capacity to support donor allogeneic BM restriction. Therefore, RAG mice may represent a model whose lymphopoietic microenvironments are functionally frozen at a fetal developmental stage, as has been suggested by thymocyte phenotype. If RAG mice represent a “fetal” model, then selection onto BM derived cells may be a normal event in the thymus, and this phenomenon may not have been routinely detected in other systems due to the use of the SCID mouse, or due to secondary effects of irradiation.

6.2 Development of Transgenic Mice Expressing Human Cytokine Genes

Based on both animal and in vitro studies, the following set of transgenes either play a role in hematopoiesis, are expressed in the bone marrow or thymus, and/or the murine proteins show specificity for murine vs. human cells: IL-3, IL-6, IL-7, M-CSF, GM-CSF, stem cell factor, LIF and oncostatin M. Genomic clones for this set of human genes were obtained and used to select ES cell clones to derive transgenic mice.

6.2.1 Isolation of Genomic Clones

PCR primer sets were designed against either the 5′ or the 3′ end of genomic sequences so that constructs containing the genes could be readily identified by PCR. In addition, these primer sets were designed to distinguish between mouse and human genes. The following primers and conditions were used to identify the human clones:

| | |
|---|---|
| human IL-7 | |
| 3181-SP6-F2: 5′ AAATCAAGCTTGAATGACAAACTCC 3′ | (SEQ ID NO:1) |
| 3181-SP6-R2: 5′ GGACAGCATGAAAGAGATTGGAGC 3′ | (SEQ ID NO:2) |
| product size: 121 bp | |
| annealing temperature: 60° C. | |
| human SCF | |
| 20180-T7-F: 5′ ATGCAAGCTTGATTCATCCTC 3′ | (SEQ ID NO:3) |
| 20180-T7-R: 5′ CGTGGTTTTTATTCGAAATGC 3′ | (SEQ ID NO:4) |
| product size: 176 bp | |
| annealing temperature: 60° C. | |
| human LIF | |
| hLIF-3F: 5′ TTCCTCTGGGTAAAGGTCTGTAAG 3′ | (SEQ ID NO:5) |
| hLIF-3R: 5′ TCCACTTGTAACATTGTCGACTTC 3′ | (SEQ ID NO:6) |
| product size: 388 bp | |
| annealing temperature: 60° C. | |
| human GM-CSF | |
| GMCSF2/3F: 5′ CTCAGAAATGTTTGACCTCCAG 3′ | (SEQ ID NO:7) |
| GMCSF2/3R: 5′ GTCTGTAGGCAGGTCGGCTC 3′ | (SEQ ID NO:8) |
| product size: 729 bp | |
| Annealing temperature: 60° C. | |
| human M-CSF | |
| 31HU-MCSF-F: 5′ GAAGACAGACCATCCATCTGC 3′ | (SEQ ID NO:9) |
| 31HU-MCSF-R: 5′ TGTAGAACAAGAGGCCTCCG 3′ | (SEQ ID NO:10) |
| product size: 401 bp | |
| Annealing temperature: 60° C. | |
| human IL-6 | |
| 51-BSF2-F: 5′ TGGTGAAGAGACTCAGTGGC 3′ | (SEQ ID NO:11) |
| 51-BSF2-R: 5′ TACTTCAAGGCGTCTCCAGG 3′ | (SEQ ID NO:12) |
| product size: 225 bp | |
| annealing temperature: 60° C. | |

Human genomic P1(for IL-6, M-CSF, and LIF), BAC(for IL-7 and SCF), and PAC(for GM-CSF) libraries (Genome Systems, Inc.) were screened using the gene specific primer sets, above. IL-3 and OM are closely lined to GM-CSF and LIF, respectively, and were not screened for in the first round. Positive clones were further confirmed by either other primer sets for the same gene or by southern blot. The following primers were used for PCR confirmation:

| | |
|---|---|
| human IL-7 | |
| 51 IL7F: 5′ GGCGTTGAGAGATCATCTGG 3′ | (SEQ ID NO:13) |
| 51 IL7R: 5′ TGCAGCTGGTTCCTCTTACC 3′ | (SEQ ID NO:14) |
| product size: 342 bp | |
| Annealing temperature: 60° C. | |
| FIL7: 5′ CATACAGCATTACAAATTGC 3′ | (SEQ ID NO:15) |
| RIL-7: 5′ TGTAGATTCTGGCCTGC 3′ | (SEQ ID NO:16) |
| product size: 322 bp | |
| annealing temperature: 60° C. | |
| human SCF | |
| SCF-DF: 5′ CCAAACTTCTGGGGCATTTA 3′ | (SEQ ID NO:17) |
| SCF-DR: 5′ CTCTCCACTGTCCCTGCTTC 3′ | (SEQ ID NO:18) |
| product size: 220 bp | |
| annealing temperature: 60° C. | |
| SCF-3F2: 5′ GCATGGAGCAGGACTCTATT 3′ | (SEQ ID NO:19) |
| SCF-3R4: 5′ AGTTTGTATCTGAAGAATAAAGCTAGG 3′ | (SEQ ID NO:20) |
| product size: 160 bp | |
| annealing temperature: 60° C. | |
| human LIF | |
| hLIF-3F: 5′ TTCCTCTGGGTAAAGGTCTGTAAG 3′ | (SEQ ID NO:21) |
| hLIF-3R: 5′ TCCACTTGTAACATTGTCGACTTC 3′ | (SEQ ID NO:22) |
| product size: 388 bp | |
| annealing temperature: 60° C. | |
| human OM | |
| OSM5F1: 5′ CCTAAAGTGAGGTCACCCAGAC 3′ | (SEQ ID NO:23) |
| OSM5R1: 5′ CTCTGTGGATGAGAGGAACCAT 3′ | (SEQ ID NO:24) |
| product size: 456 bp | |
| annealing temperature: 60° C. | |
| OSM3F1: 5′ GAGATCCAGGGCTGTAGATGAC 3′ | (SEQ ID NO:25) |
| OSM3R1: 5′ GATGCTGAGAAGGGGAGAGAG 3′ | (SEQ ID NO:26) |
| product size: 384 bp | |
| annealing temperature: 60° C. | |
| human GM-CSF | |
| GMCSF1/2F: 5′ AGCCTGCTGCTCTTGGGCAC 3′ | (SEQ ID NO:27) |
| GMCSF1/2R: 5′ CTGGAGGTCAAACATTTCTGAG 3′ | (SEQ ID NO:28) |
| product size: 282 bp | |
| annealing temperature: 60° C. | |
| GMCSF3/4F: 5′ ATGGCCAGCCACTACAAGCAG 3′ | (SEQ ID NO:28A) |
| GMCSF3/4R: 5′ GGTGATAATCTGGGTTGCACAG 3′ | (SEQ ID NO:29) |
| product size: 878 bp | |
| annealing temperature: 60° C. | |
| human IL-3 | |
| IL-3F: 5′ CGTCTGTTGAGCCTGCGCAT 3′ | (SEQ ID NO:29A) |
| IL-3R: 5′ AAATCTCCTGCCATGTCTGCC 3′ | (SEQ ID NO:29B) |
| product size: 298 bp | |
| annealing temperature: 60° C. | |
| human M-CSF | |
| HUM-CSF-5F1: 5′ GAGGGAGCAAGTAACACTGGAC 3′ | (SEQ ID NO:30) |
| HUM-CSF-5R1: 5′ CGTCTTCCTAGTCACCCTCTGT 3′ | (SEQ ID NO:31) |
| product size: 322 bp | |
| annealing temperature: 60° C. | |
| human IL-6 | |
| IL6-3F: 5′ CTAGATGCAATAACCACCCCTG 3′ | (SEQ ID NO:32) |
| IL6-3R: 5′ CAGGTTTCTGACCAGAAGAAGG 3′ | (SEQ ID NO:33) |
| product size: 217 bp | |
| annealing temperature: 60° C. | |

Plasmid DNA from P1, BAC, or PAC clones was prepared using the KB-100 Magnum columns (Genome Systems, Inc.). Detailed experimental procedures were described in detail in the user's manual supplied by the manufacturer. To quantify DNA concentrations, DNA constructs were digested with EcoRI, followed by electrophoresis on 0.8% agarose gel along with DNA standards with known concentration. Plasmid DNA concentrations were determined by comparison with the standards.

The following DNA constructs were identified as having the full structural sequences for the target genes based upon the presence of both 5′- and 3′- ends of the coding regions:

| | |
|---|---|
| IL-7: | BAC20854 (100 kb), BAC2267C7 |
| | (110 kb), PAC24404 (90 kb) |
| SCF: | BAC21029 (145 kb) |
| LIF: | P1-20872 (100 kb), P1-20873 (100 kb) |
| GM-CSF: | PAC21689 (150 kb), PAC21691 (194 kb) |
| M-CSF: | P1-3882 (55 kb) |
| IL-6: | P1-3877 (n/d), P1-3878 (65 kb) |

The sizes of the clones (in parentheses) were determined by restriction digestion with NotI followed by pulse-field gel electrophoresis. The gel running conditions were set as followed:

initial switch time: 1 sec

final switch time: 6 sec

total run time: 12 hrs

voltage: 6 v/cm

angle: 120° C.

6.2.1 Demonstration of In Vitro Transcription from the Genomic Constructs

To determine the ability of the human genomic clones to be transcribed by murine cells, undigested genomic constructs were transfected into MM54 cells (a murine cell line that expresses the endogenous form of each cytokine, ATCC # 6434-CRL) by lipofection using Tfx50 (Promega) according to the manufacturer's instructions. The cells were harvested 48 hours later for mRNA analysis. Total RNA was prepared using the Ultrspec™ RNA isolation system (Biotecx). 10 mg of gelatin carrier protein was added prior to ethanol precipitation to enhance RNA yield. First strand cDNA was synthesized by standard reverse transcription methods. Briefly, the RNA was resuspended in 29.5 ml of H₂O, and mixed with 10 ml 5× first strand buffer, 2.5 ml 10 mM dNTP, 5 ml 0.1M DTT, 1 ml 0.5 mg/ml random primer, and 2 ml M-MLV reverse transcriptase (Life Technologies). The reaction was incubated at 37° C. for 1 hour, and the cDNA was purified by Phenol/Chloroform extraction. The resulting cDNA was then resuspended in 20 ml of H₂O. Human specific transcripts were analyzed by nested-PCR methods. 1 ml of cDNA sample was first amplified with the first PCR primer-set for 30 cycles. After that, a 5 μml aliquot was taken from the reaction mixture and subjected to a second round of PCR with the nested PCR primer set for an additional 30 cycles. The DNA samples were resolved on a 1% agarose gel.

Primer sets for nested-PCR:

| | |
|---|---|
| human IL-7 | |
| 1St round primers (Clontech): | |
| CTIL-7F: 5′ ATGTTCCATGTTTCTTTTAGGTATATCT 3′ | (SEQ ID NO:35) |
| CTIL-7R: 5′ TGCATTTCTCAAATGCCCTAATCCG 3′ | (SEQ ID NO:36) |
| product size: 681 bp | |
| annealing temperature: 60° C. | |
| 2nd round primers: | |
| hIL-7F1: 5′ GCATCGATCAATTATTGGACAGC 3′ | (SEQ ID NO:37) |
| hIL-7R1: 5′ CTCTTTGTTGGTTGGGCTTCAC 3′ | (SEQ ID NO:38) |
| product size: 280 bp | |
| annealing temperature: 60° C. | |
| human SCF | |
| 1st round primers: | |
| hSCF5F3: 5′ CACTGTTTGTGCTGGATCGCAG 3′ | (SEQ ID NO:39) |
| hSCFB-R: 5′ TGAGACACGTGCTTTCTCTTCC 3′ | (SEQ ID NO:40) |
| product size: 1173 bp | |
| annealing temperature: 60° C. | |
| 2nd round primers: | |
| hSCF3F1: 5′ CAGCCAAGTCTTACAAGGGCAG 3′ | (SEQ ID NO:41) |
| hSCFA-R: 5′ AGACCCAAGTCCCGCAGTCC 3′ | (SEQ ID NO:42) |
| product size: 364 bp | |
| annealing temperature: 60° C. | |
| human LIF: | |
| 1st round primers: | |
| hLIF-F1: 5′ TAATGAAGGTCTTGGCGGCAGGAG 3′ | (SEQ ID NO:43) |
| hLIF-R2: 5′ TCCTGAGATCCCTCGGTTCACAGC 3′ | (SEQ ID NO:44) |
| product size: 652 bp | |
| annealing temperature: 60° C. | |
| 2nd round primers: | |
| hLIF-F2: 5′ AACAACCTCATGAACCAGATCAGGAGC 3′ | (SEQ ID NO:45) |
| hLIF-R1: 5′ ATCCTTACCCGAGGTGTCAGGGCCGTAGG 3′ | (SEQ ID NO:46) |
| product size: 402 bp | |
| annealing temperature: 60° C. | |
| human GM-CSF: | |
| 1st round primers (from Clontech): | |
| CT-hGMCSF-F: 5′ ATGTGGCTGCAGAGCCTGCTGC 3′ | (SEQ ID NO:47) |
| CT-HGMCSF-R: 5′ CTGGCTCCCAGCAGTCAAAGGG 3′ | (SEQ ID NO:48) |
| product size: 424 bp | |
| annealing temperature: 600 C. | |
| 2nd round primers: | |
| hGMCSF-F1: 5′ CGTCTCCTGAACCTGAGTAGAG 3′ | (SEQ ID NO:49) |
| hGMCSF-R1: 5′ CAAGCAGAAAGTCCTTCAGGTTC 3′ | (SEQ ID NO:50) |
| product size: 276 bp | |
| annealing temperature: 60° C. | |
| human IL-6: | |
| 1st round primers (from Clontech): | |
| CT-hIL6F: 5′ ATGAACTCCTTCTCCACAAGCGC 3′ | (SEQ ID NO:51) |
| CT-hIL6R: 5′ GAAGAGCCCTCAGGCTGGACTG 3′ | (SEQ ID NO:52) |
| product size: 628 bp | |
| annealing temperature: 60° C. | |
| 2nd round primers: | |
| hIL6-F2: 5′ TGGGGCTGCTCCTGGTGTTGC 3′ | (SEQ ID NO:53) |
| hIL6-R2: 5′ CAGGAACTCCTTAAAGCTGCG 3′ | (SEQ ID NO:54) |
| product size: 560 bp | |
| annealing temperature: 60° C. | |
| human M-CSF: | |
| 1st round primers: | |
| hMCSF-F: 5′ CTCTCCCAGGATCTCATCAGCG 3′ | (SEQ ID NO:55) |
| hMCSF-R1: 5′ CAGGATGGTGAGGGGTCTTAG 3′ | (SEQ ID NO:56) |
| product size: 492 bp | |
| annealing temperature: 60° C. | |
| 2nd round primers: | |
| hMCSF-F: 5′ CTCTCCCAGGATCTCATCAGCG 3′ | (SEQ ID NO:57) |
| hMCSF-R2: 5′ TTGCTCCAAGGGAGAATCCGCTC 3′ | (SEQ ID NO:58) |
| product size: 410 bp | |
| annealing temperature: 60° C. | |

The following genomic clones produced human-specific transcripts and were chosen for use in ES cell transfection:

IL-7: BAC20854

SCF: BAC21029

LIF: P1-20872, P120873

GM-CSF: PAC21689, PAC21691

M-CSF: P1-3882

IL-6: P1-3878

6.2.2 Selection of ES Clones that Contain Human Cytokine Genes

Murine embryonic stem (ES) cells (either RAG−/− ES cells or 129Sv/J wild type ES cells) were transfected with 3 sets of genes of human hematopoietic growth factors. The liposome reagent, Tfx-50 (Promega) was used according to the manufacturer's instructions. Each set of genes contained equal molar concentration of 3 linearized growth factor DNA. The first DNA set (GM-CSF set) contained GM-CSF, M-CSF and IL-6. The second DNA set (IL-7 set) contained IL-7, SCF and LIF. The third DNA set contained all 6 transgenes. Plasmid DNA with a selectable marker, either PGK-Hyg (for RAG−/− ES cells) or PGK-Neo (for 129Sv/J wild type ES cells), was used for positive selection.

Briefly, the growth factor DNA was linearized by digestion with Not1. The growth factor DNA mixtures (2.18 mg) and linearized selectable marker DNA (0.42 μg) was mixed in 1 ml serum free Opti-MEM media and incubated with 170.6 μg (97.5 ml) Tfx-50 for 15 minutes at room temperature. The molar ratio of marker versus DNA mixture was 4:1 and the ratio of Tfx-50 versus total DNA (marker and growth factor DNA) was 25:1. Then, 6-9×10⁶ ES cells in 5 ml of serum free Opti-MEM media were added to the DNA/liposome mixture and incubated for 1 hr at 37° C. After 1 hr incubation, the cells were harvested and replated in 6 well plates at a concentration of 2.5×10⁵ ES cells per well. Hygromycin (120 μg/ml) or G418 (400 μg/ml) selection was started 24 hrs post transfection. Drug resistant ES colonies were picked after 10 to 14 days of selection.

6.2.3 Southern Blot Analysis of Transgenic ES Clones and Determination of Gene Copy Numbers

All DNA probes for the genes were generated by PCR from either human genomic DNA or cDNA samples, then cloned into the pCR^{R}2.1-TOPO vector. The PCR fragments were then recovered from the plasmid by EcoRI digestion and gel purification using, e.g., a Gel Extraction Kit (Qiagen).

human IL-7: A 350 bp genomic fragment was amplified from total human DNA with primer set 51 IL7F/51 IL7R (SEQ ID NOs:13 and 14).

human SCF: A 1173 bp cDNA fragment was amplified from cDNA extracted from human embryonic kidney cell line 293 with primer set hSCF5F3/hSCFB-R (SEQ ID NOS:39 and 40). This fragment was subsequently cloned into the pCRR2.1-TOPO vector. After EcoRI digestion, an 808 bp DNA fragment was purified from the gel and was used as the probe for identifying SCF.

human LIF: The 388 bp PCR fragment(hLIF-3F/hLIF-3R) was subcloned and used as the probe (hLIF-3F/hLIF-3R; SEQ ID NO:21 and 22).

human GM-CSF: The 424 cDNA fragment was generated by PCR with primer set CT-hGMCSF-F/CT-hGMCSF-R (SEQ ID NO:47 and 48) from human 293 cell cDNA samples.

human M-CSF: The 400 bp probe was generated with PCR primer set 31HU-MCSF-F/31HU-MCSF-R (SEQ ID NO:9 and 10).

human IL-6: The 298 bp probe was generated with primer set 51-BSF2-F/51-BSF2-R (SEQ ID NO: 11 and 12).

ES cell clones were analyzed by Southern blotting to confirm the presence of genomic sequences and to determine relative copy number in comparison to human DNA controls. 10 μg of DNA from each ES cell clones was digested with either EcoRI (for IL-7 and SCF), BamHI (for LIF and IL-6), or HindIII (for GM-CSF and M-CSF) and resolved on 1% agarose gel. The DNA was transferred to a nylon membrane by alkaline transfer (user's manual, Genescreen Plus). The membranes were then prehybridized over night at 420 C. with standard formamide containing buffer (Ausubel). Each probe was labeled using the Prime-It II Kit (Stratagene) and then added to the membrane. The hybridizations were carried out overnight with rotation at 420 C. The membranes were washed two times at room temperature with the low stringency buffer (2×SSC, 0.1% SDS) for 10 min each, and two more times at 500 C. for 10 min each. The membranes were then dried by blotting in between two layers of Whatman paper, and exposed to phospho screens (Molecular Dynamics). The image was quantified by the STORM System (Molecular Dynamics). The copy number for each transgene was derived by comparison with the human control.

Over 3400 drug resistant colonies were picked, of these, 264 clones have been expanded. From these clones, 179 ES clones were found suitable for injection. Among these injectable ES clones, 2 had 6 genes, 18 had 5 genes and 95 clones had 3 transgenes.

The copy number for the same gene varied among different clones. For example, clone 6 had one copy of IL-6 gene whereas clone 15 had two copies of the same gene. On the other hand, within the same clone, the copy number of one gene varied from the other gene. For example, clone 18 had one copy of IL-7 gene, two copies of SCF gene, and three copies of LIF gene.

6.2.4 Generation of Transgenic Mice

ES cell clones containing the human cytokine genes are used to derive transgenic mice as described in Robertson (ed), Teratocarcinomas and embryonic stem cells—a practical approach (1987), IRL Press. ES cells are injected into 3.5 day p.c. C57BL/6 embryos and implanted into the uterus of pseudopregnant females and allowed to develop to birth. Male chimeras are mated with wild type C57B1/6 females to obtain germline transgenic lines.

6.2.5 Identification of Mice with Human Transgenes

There are two ways to identify mice that have incorporated human transgenes: Southern Blot and PCR analysis. It is preferable to use PCR to genotype the mice due to its speed and ease of experimental procedure. However, whenever there is concern about the validity of PCR results, Southern Blot should be carried out to confirm the results. Briefly, DNA samples were isolated from the tips of mice tails following standard protocols (Qiagen manual, DNeasy 96 Tissue Kit). PCR analysis using human-specific primer sets (see Section 6.2.1) was performed for each transgene. A positive control sample containing human DNA and a negative control sample containing mouse DNA were also carried out at the same time to ensure the specificity of the PCR products. Only mice that contain the expected human transgenes were selected for further breedings and experiments.

Seven independent lines of transgenic mice have been established so far. Clone 12 and clone 71 have IL-6, M-CSF, and GM-CSF. Clone 74 and clone 75 have IL-7, SCF, and LIF. Clone 182 and clone 185 have all six transgenes in the germline, whereas clone 201 has every gene except for LIF. The same procedure was done throughout the breeding process to ensure the genotypes of the mice.

6.2.6 Demonstration of In Vivo Transcription from the Genomic Constructs

Total RNA samples were prepared (RNeasy Midi Kit, Qiagen) from nine tissues of each transgenic mouse, including spleen, thymus, liver, kidney, heart, muscle, lung brain, and bone marrow. Total cDNA was prepared as previously described (see Section 6.2.1). Gene expression analysis for each transgene was carried out by nested-PCR (see above). To ensure the reproducibility of the results, at least two mice from each genotype were analyzed by this method.

Mice from clone 71, which have human IL-6, M-CSF, and GM-CSF, showed expression of all three transgenes in different tissues. Human IL-6 was mainly expressed in the spleen and thymus. Human GM-CSF expression was restricted in the thymus. On the other hand, human M-CSF has a much wider tissue distribution, with transcripts in the spleen, thymus, liver, kidney, heart, muscle, lung, and brain. Mice from clone 75, which have human IL-7, SCF, and LIF, also showed expression of all three transgenes in tissues. Human IL-7 and SCF seem to have a wide distribution pattern similar to M-CSF, whereas human LIF expression was restricted to the brain.

Mice from other ES clones that contain either IL-6, M-CSF, and GM-CSF, or IL-7, SCF, and LIF were also analyzed by the same method. Although the expression pattern vary in certain tissues, the overall pattern was similar. This variation may be attributed to the difference in the insertion site of the transgenes and the copy numbers for each gene. The murine endogenous genes were also analyzed by nested-PCR. Despite differences in certain specific tissues, the expression pattern largely agrees with that of the human transgenes.

Protein expression of human transgenes in serum and in the supernatant of bone marrow stromal cell cultures derived from the injected mice were examined by ELISA.

It was found that transgene expression patterns and levels varied between clones, litters, littermates, and even stromal cells from same mouse. The following table provides ELISA results from mice injected with 4 ES cell clones.

TABLE 3
Transgene Expression Patterns

| | | GM-CSF set | | | | | |
|---|---|---|---|---|---|---|---|
| | | of transgenes | | | IL-7 set of transgenes | | |
| ES clone | Media | M-CSF | IL-6 | GM-CSF | SCF | IL-7 | LIF |
| clone 12 | Serum | X | — | — | N/A | N/A | N/A |
| | Super- | — | — | — | N/A | N/A | N/A |
| | natant | | | | | | |
| clone 71 | Serum | X | X | — | N/A | N/A | N/A |
| | Super- | X | X | — | N/A | N/A | N/A |
| | natant | | | | | | |
| clone 74 | Serum | N/A | N/A | N/A | X | — | — |
| | Super- | N/A | N/A | N/A | — | — | — |
| | natant | | | | | | |
| clone 75 | Serum | N/A | N/A | N/A | X | X | — |
| | Super- | N/A | N/A | N/A | — | X | — |
| | natant | | | | | | |

In addition to transgene transcription and translation, the ability of the stromal cells to support hematopoietesis was investigated.

To examine the effects of transgenic murine hematopoietic microenvironment on human hematopoiesis, long term bone marrow cultures derived from transgenic or wildtype littermates were set up in tissue culture flasks.

After 2 weeks of culture, a monolayer of bone marrow stromal cells will form and adhere to the bottom of flasks. Hematopoietic stem/progenitor cells then adhere to the stromal layer. As hematopoietic cells proliferate and differentiate, they become non-adherent and float freely in the supernatant of the culture. The cell number of differentiated cells can be counted and stained to determine the extent of proliferation and differentiation of the hematopoietic stem cells.

Once a stromal layer formed, the cultures were irradiated to eliminate murine hematopoiesis and to stop proliferation of stromal cells. Irradiation, however, maintained the ability of the stromal cells to support human hematopoiesis in vitro. After irradiation, human cord blood mononuclear cells were added to the culture. Cell counts were made weekly, as was a 50% change in media. Every week, the non-adherent cells were counted and analyzed by FACS.

Stromal cells from clone 71 transgenic mice supported human hematopoiesis in vitro better than clone 12 and clone 75. Non-adherent cells harvested from transgenic co-cultures were greater in number than wild type co-cultures established from clone 71 littermates. Mixtures of stromal cells from clone 71 and 75 transgenic mice supported human hematopoiesis in vitro better than stromal cells from either clone 71 or clone 75 alone, in terms of non-adherent cellularity.

The effects of human transgenes on murine hematopoiesis were also examined. Expression of human transgenes increased bone marrow B cell progenitor production in transgenic littermates of clone 71 and 75 mice.

6.3 Ability of Irradiated H-2^{D} [symbol] H-2^{B} C1D/RAG-2 and H-2^{B} C2D/RAG-2 Bone Marrow Chimeras to Support Functional Engraftment

MHC class I deficient (C1D)/RAG-2 and class II deficient (C2D)/RAG-2 mice were tested to assess whether MHC was necessary to facilitate alloengraftment. Unirradiated allogeneic C1D/RAG-2 chimeras produced antigen specific IgG antibody when chimeras contained greater than 10% donor B lymphocytes in the peripheral blood. In comparison, irradiation (800 rads) of C1D/RAG-2 hosts led to a relative increase in the levels of donor cell engraftment, with a higher percentage of B cells in peripheral blood. All of these chimeras produced good antigen specific IgG antibody to KLH. Although radiation conditioning was not found to be an absolute requirement for the functional engraftment of allogeneic C1D/RAG-2 chimaeras, irradiated hosts supported more extensive cellular and functional engraftment.

In contrast to allogeneic C1D/RAG-2 chimeras, unirradiated C2D/RAG-2 mice were unable to support cellular alloengraftment, therefore irradiation preconditioning was used. The level of thymocyte development in H-2^{d} [symbol] H-2^{b} C2D/RAG-2 mice that received 800R irradiation was significantly better. The relative percentage of CD4⁺ cells was diminished relative to C1D/RAG-2 chimeras, which correlated with the absence of host-expressed MHC Class II molecules, but CD4 development was present. These chimaeras elicited an anti-KLH antibody response following immunization, demonstrating the functional engraftment of these mice. This suggests that neither class I nor class II are absolutely required in the recipient for functional engraftment of RAG-2 mice.

6.3.1 Evaluating the MHC Haplotype Dependence in Supporting Donor MHC-Restricted Immunity

The following experiments were performed and conclusions were drawn that the RAG-2 mutation confers a “universal” property to support the functional development of allogeneic HSC.

The following allogeneic and syngeneic bone marrow chimaeras were prepared:

(i) Balb/c (H-2^{d}) [symbol] Balb/c RAG-2 (H-2^{d}) hosts

(ii) C57B1/6 (H-2^{b}) [symbol] Balb/c RAG-2 (H-2^{d}) hosts

(iii) Balb/c (H-2^{d}) [symbol] 129 RAG-2 (H-2^{b}) hosts

(iv) C57B1/6 (H-2^{b}) [symbol] 129 RAG-2 (H-2^{b}) hosts

(v) Balb/c (H-2^{d}) [symbol] C57B1/6 SCID (H-2^{b}) hosts

(vi) C57B1/6 (H-2^{b}) [symbol] C57B1/6 SCID (H-2^{b}) hosts

The first set of chimeras (i-iv) were designed to determine whether RAG-2 mutant mice on an H-2^{d} background have the ability to support allogeneic donor-specific immunity. All of these chimeras supported functional engraftment.

The second set of chimeras (v-vi) were prepared to test the ability of SCID mutant mice, on an H-2^{b} background, to support allogeneic donor-specific immunity. The allogeneic chimeras engrafted very poorly relative to the syngeneic group (thymuses were too small to sample) which is very similar to the H-2^{d} into H-2^{b} SCID results reported. These results suggest there is a significant difference between the SCID and RAG-2 mutations to support the cellular development of T lymphocytes independent of MHC haplotype.

To assess whether RAG-2 hosts could support functional engraftment from a donor with an unrelated haplotype, an H-2^{k} AKR [symbol] H-2^{b} RAG-2 chimera was produced. These mice supported donor-derived immunity.

Taken together, these results indicate the RAG-2 mutation supports bone marrow alloengraftment from different donor strains, independent of haplotype. This suggests these mice could support hematopoiesis from any donor.

6.3.2 Evaluating Other Mutations for Donor-Derived Immunity

RAG-2 mice appeared to be unique relative to other immunodeficient strains in supporting donor-restricted immunity until transplantation studies in RAG-I and TCR/(with irradiation to eliminate host B cells) mice were performed. Although unirradiated RAG-1 chimaeras did not support engraftment from allogeneic donors, irradiated (800 rads) RAG-1 mice supported functional engraftment. Both RAG-1 and RAG-2 genes are required to initiate T and B lymphocyte receptor rearrangements. In addition, studies showed that irradiated TCR/mice also supported donor-derived immunity.

6.3.3 Cytochrome-C Specific TCR Transgenic (_{H-2K} Class II-Restricted) [symbol] H-2B RAG-2 Bone Marrow Chimaeras Support Cellular Engraftment of Donor CD4⁺ T Cells in the Absence of Host Expression of Cognate MHC Class II Molecules

In order to determine the mechanism of donor-derived immunity, SJL-TgN(TcrAND)53Hed mice were obtained from Jackson Laboratory and backcrossed onto AKR (H-2^{k}) mice to provide the appropriate MHC Class II molecule (I-E^{k}) for positive selection of TCR-transgenic (TCR-tg) T cells (which recognize cyt-c in the context of (I-E^{k}). Bone marrow from these mice was used to engraft H-2^{b} RAG-2 mice which do not express the cognate MHC Class II receptor for the transgenic T cells. This created a host environment for the transgenic bone marrow cells that is functionally equivalent to a Class II knockout background. Donor T cell development would therefore be dependent on donor-derived antigen presenting cells to positively select TCR-tg T cells.

The percentages of thymocytes in TCRtgxAKR [symbol] RAG-2 chimaeras were similar to that of wild type AKR [symbol] RAG-2 mice. Both of these chimeras have overall lower percentages of T cells in comparison to TCRtgxAKR donor mice consistent with other haplotype combinations of allogeneic RAG-2 chimaeras. The level of B cell reconstitution was relatively high (greater than 10%). Both the TCRtgxAKR [symbol] RAG-2 and AKR [symbol] RAG-2 were immunized with cytochrome c to determine their ability to produce antigen-specific IgG antibody.

6.4 Development of Transgenic Mice Expressing Human HLA Class II Genes

An alternative embodiment of the invention involves the expression of human HLA Class II molecules in MHC Class II-bearing tissues of the mouse. In this example, donor HSC are introduced that express the same HLA haplotype(s) as the transgenic HLA Class II molecules. This combination provides cognate interactions between donor T lymphocytes, which develop in the context of the transgenic HLA Class II molecules expressed on the host tissues (in particular the thymus), and donor-derived B lymphocytes. Although the methods for making transgenic mice that express human HLA Class II molecules of the DR3 haplotype are taught, these methods can be applied to any desired HLA haplotype, including those for Class I genes, for the purpose of evaluating responses representing other individuals in the population.

6.4.1 Preparation of YAC DNA for Lipofection

YAC 4D1 spans approximately 550 kb of the HLA Class II region (Ragoussis et al. Nucleic Acids Research, 20:3135-3138 (1992), and Ragoussis, et al. in Tsuji, et al. (eds.) HLA 1991, Oxford Univ. Press (1992)). It is bordered on one end by the RING3 gene, and the opposite end by DRa. It contains the DRa, DRb, DQa, and DQb chains of the DR3 haplotype.

Yeast cultures containing the 4D 1 YAC were grown in AHC media. Agarose blocks were formed in 1% low melting temperature agarose containing approximately 3×10⁹ cells/ml. The YAC was separated from yeast chromosomes by pulse-field gel electrophoresis in a 1% low melting temperature agarose gel. Running conditions were: 200V, 40 hours duration, with a 50 second switch time. After electrophoresis, the gel was cut lengthwise at the outer edges and in the middle. The three slices were stained with ethidium bromide to visualize the position of the 4D 1 YAC vis a vis the host chromosomes. The position of the 4D1 YAC was marked with notches and the marker pieces realigned with the unstained gel sections. A horizontal band containing the 4D1 YAC was excised based on the position of the notches.

The 4D1 gel slices were equilibrated twice for one hour/each in 1× gelase buffer (Epicentre Technologies) on a rotating platform. The buffer was changed after the second rinse and left at 4° C. overnight. Based on the input amount of yeast DNA, the estimated amount of 4D1 DNA in the entire gel was approximately 8 mg. The following day, the gel slices were cut into 20 blocks weighing approximately one gram each, and placed into individual tubes. The gel fragments were melted at 70° C. for 20 minutes and then equilibrated at 45° C. for 15 minutes. Ten units of Gelase (Epicenter Technologies, 1 unit/ml) was added per tube and incubated at 45 oC. for 45 minutes. The gelase step was then repeated.

6.4.2 Transfection of YAC DNA into ES Cells

Each agarose block contained approximately 400 ng of YAC DNA, or 400 ng/ml. A neomycin resistance plasmid (PGKneo) was added at a molar ratio of approximately 4:1 (20 ng per ml of gel block). Transfectam (Promega, lot 318402) was added at a 50:1 weight:weight ratio (approximately 19 mg per ml of gel block) and the mixture allowed to sit at room temperature for one hour. ES cells had been split 1:2 the day before and seeded onto 100 mm plates. The cells were trypsinized on the day of transfection and resuspended at 3×106 cells/ml in serum-free ES media. One ml of the ES cells was placed into 60 mm dishes with eight ml of serum-free ES media. One ml of DNA/lipid mixture was added and the cells were incubated at 37° C. for 4 hours. Afterward, the lipofection/ES cell mixture was plated onto feeder cells at 1×10⁶ ES cells per 100 mm dish. G418 [400 mg/ml] was added to the media the following day and changed every other day for 9-12 days until clones appeared. Individual clones were picked and grown in 96 wells. The cells were split 1:2 into duplicate 96 well plates. One plate was frozen in situ and the other was harvested for DNA analysis.

6.4.3 Characterization of 4D1-Positive Clones

The presence of the entire YAC was determined using PCR primers for six genes that span the entire 550 kb: TAP-1, TAP-2, DQb, DQa, DRb, and DRa. The first screen involved the TAP-1 and DRa primer sets. Clones that were double positive for these two end-region genes were further screened with the remaining four primer sets.

| | |
|---|---|
| Tap 1: | |
| 1069 F: CAC CCT GAG TGA TTC TCT | (SEQ ID NO:59) |
| 1069 R: ACT GAG TCT GCC AAG TCT | (SEQ ID NO:60) |
| Tap 2: | |
| 1231 F: GCG GAG AGA CCT GGA ACG | (SEQ ID NO:61) |
| 1231 R: TCA GCA TCA GCA TCT GCA | (SEQ ID NO:62) |
| DQα: | |
| GH26: GTG CTG CAG GTG TAA ACT TGT ACC AG | (SEQ ID NO:63) |
| GH27: CAC GGA TCC GGT AGC AGC GGT AGA GTT G | (SEQ ID NO:64) |
| DQβ: | |
| GH28: CTC GGA TCC GCA TGT GCT ACT TCA CCA ACG | (SEQ ID NO:65) |
| GH29: GAG CTG CAG GTA GTT GTG TCT GCA CAC | (SEQ ID NO:66) |
| DRα: | |
| DRα F: CTT TGC AAG AAC CCT TCC C | (SEQ ID NO:67) |
| DRα R: ATA GCC CAT GAT TCC TGA GC | (SEQ ID NO:68) |
| DRβ: | |
| GH46: CCG GAT CCT TCG TGT CCC CAC AGC ACG | (SEQ ID NO:69) |
| GH50: CTC CCC AAC CCC GTA GTT GTG TCT GCA | (SEQ ID NO:70) |

All product sizes are 300 bp.

Tap 1 PCR Program:

92C 15″

55C 30″

72C 1′ (30X)

Tap 2 PCR Program:

96C 20″

65C 30″

72C 30″ (30X)

(Requires 2 rounds of PCR)

DR and DQ PCR Program:

95C 15″

55C 30″

72C 1′ (30X)

6.4.4 Generation of Transgenic Mice

Clone 4D1.18 was used to derive transgenic mice as described in Robertson (ed), TERATOCARCINOMAS AND EMBRYONIC STEM CELLS—A PRACTICAL APPROACH (1987), IRL Press. ES cells were injected into 3.5 days p.c. C57BL/6 embryos and implanted into the uterus of pseudopregnant females and allowed to develop to birth. Chimeric males were mated with wild type C57B1/6 females to obtain germline transgenic lines.

6.4.5 Antibody Response of Transgenic Mice

Four D1/C2D/RAG-2 (HLA-transgenic) mice were bled and their sera tested for I-Ealpha, I-Ebeta and DRalpha expression using FACS analysis. Mice that were confirmed to express surface DR but not I-Ealpha were chosen for functional testing. These mice were immunized via the footpad with 50 μg/mouse. Three proteins were used as an immunogen. Two were fungal proteases and the third was a hybrid of the two proteins which has been found to be of reduced allergenicity in an in vitro human T cell epitope assay. The proteins were emulsed with CFA for total volume of 100 μl per footpad and boosted 2 weeks later with the same concentration in IFA in the other footpad. Immunized mice were bled 1 week later and sera samples were tested for antibodies to the appropriate protein by ELISA. A second set of animals were immunized for antibody responses but using a different protocol which is as follows: mice were immunized intraperitoneally with 50 μg/mouse of the same three proteins emulsed with CFA for total volume of 100 μl per mouse and boosted ip 2 weeks later with the same concentration in IFA. Mice were bled 1 week later and sera samples were tested for antibodies.

To assess whether the T cells in these transgenic mice were functioning normally, a positive control immunogenic peptide known to be a major T cells epitope (HSP65 1-20) was used. Mice were immunized according to a previously reported protocol by Geluk et. al. Popliteal lymph nodes were taken and T cell proliferation assessed using a T cell proliferastion assay (also reported by Geluk et. al.). A summary of the array of experiments is below.

| Protein | T Cell Response | Antibody Response |
|---|---|---|
| Protein 1 | ND | ++ |
| Protein 2 | ND | ++ |
| Hybrid protein | ND | + |
| HSP65 epitope | ++ | ND |
| Tetanus Toxoid | ND | +++++ |
| KLH | ND | +++++ |

The results suggest that the immune system in these transgenic mice is functionally intact and may be used to assess DR3-specific immune responses.

6.5 Ability of Bone Marrow Stromal Cells Obtained from I-Mune Mice to Support Human Hematopoietic Stem Cells

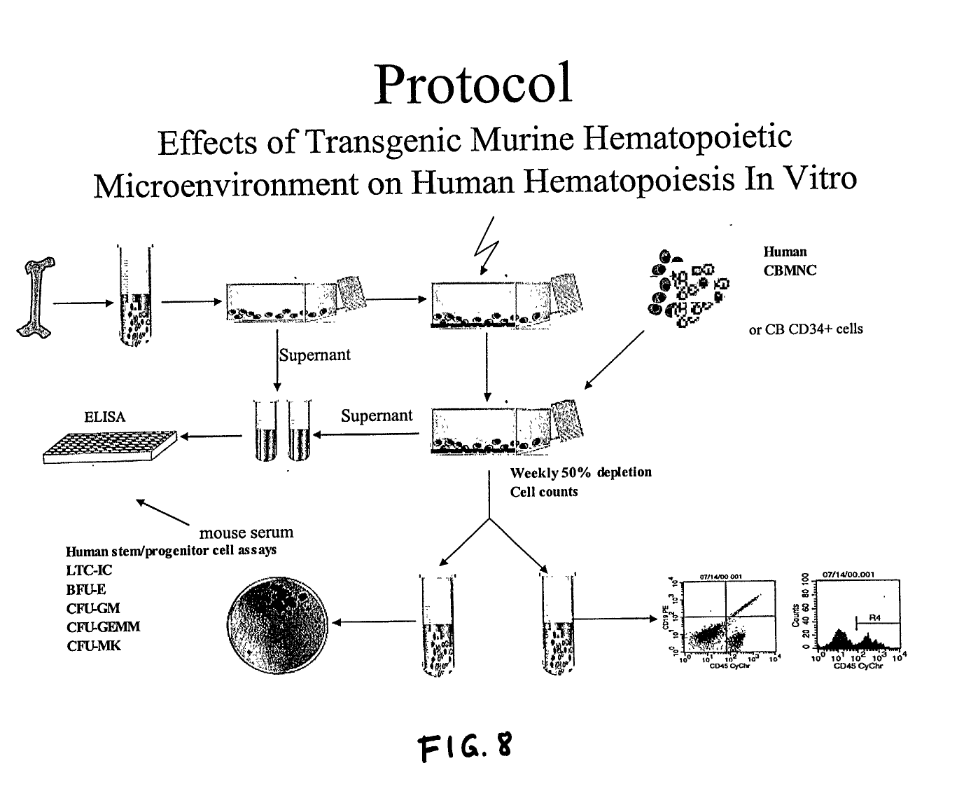

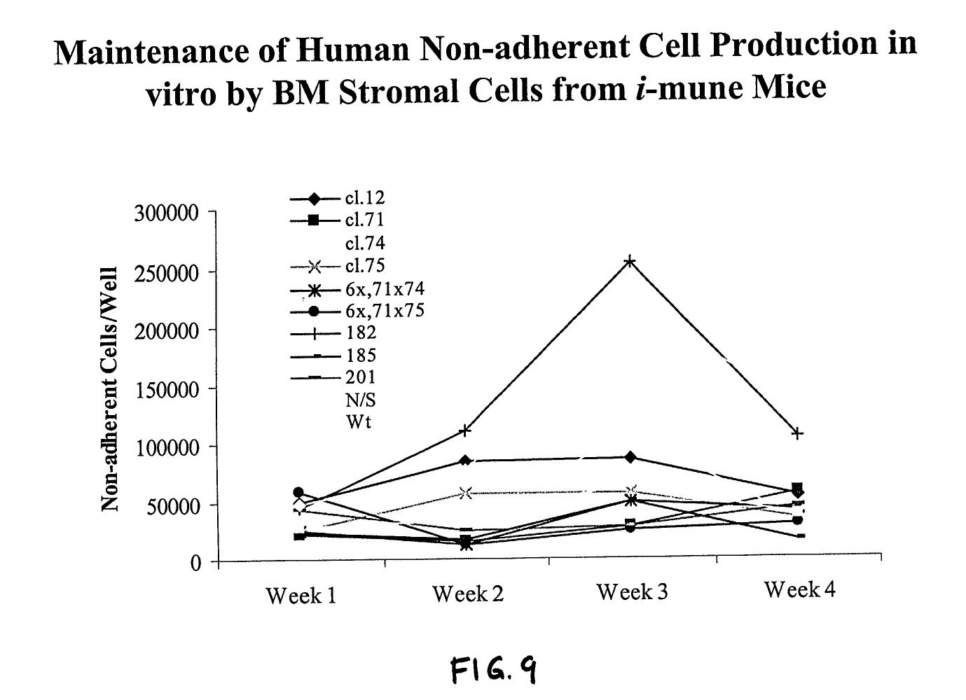

Bone marrow stromal cells were obtained from the i-mune mice of the invention, as well as from the wild-type parental strain, and long term bone marrow cultures were made. FIG. 8 sets forth schematically the methodology of making the long term bone marrow stromal cell cultures. Transgenic mice and wild type littermates were killed by cervical dislocation. Bone marrow cells (BMCs) were harvested from the hind limbs of all mice. Harvested BMCs were then counted on a Coulter Counter with a 100-μm aperture, after addition of Zapoglobin for red blood cell lysis (according the manufacturer's recommendations). Six million (6×10⁶) low-density mononuclear cells were plated per well in 6-well tissue culture plates in murine myeloid long-term culture medium (MyeloCult™ M5300, StemCell Technologies). Cultures were maintained in 33° C. in 5% CO₂. BMLTC were irradiated (30 Gy, ¹³⁷Cs at 116 cGy/min) after a confluent adherent stromal layer formed (usually 12 to 14 days after the cells were plated). BMLTC medium was then completely changed to human myeloid long-term culture medium (MyeloCult™ H5100, StemCell Technologies) and overlaid with 15×10⁶ human cord blood mononuclear cells (CBMNC). BMLTC were demidepopulated weekly, removing 50% of the media and nonadherent (NA) cells. NA cells obtained weekly from BMLTC were counted and 5×10⁴ cells were plated in 1 ml aliquots of complete methylcellulose media (MethCult™ GF H4434, StemCell Technologies). Duplicate cultures were incubated at 37° C. in 5% CO₂ in humidified 10×35 mm tissue culture dishes (Nunc Inc.) for 14 to 16 days and colonies (>50 cells) were counted on an inverted microscope and scored as colony-forming units granulocyte and macrophage (CFU-GM), burst-forming units-erythroid (BFU-E), or multilineage colony-forming units (CFU-Mix). The phenotypes of NA cells were examined by flow cytometry at various time points during the long-term culture period. The assays were performed at weeks 1, 2, 3 and 4 after seeding of the human cells onto the long term bone marrow stromal cell cultures obtained from the i-mune mice and control mouse strain.

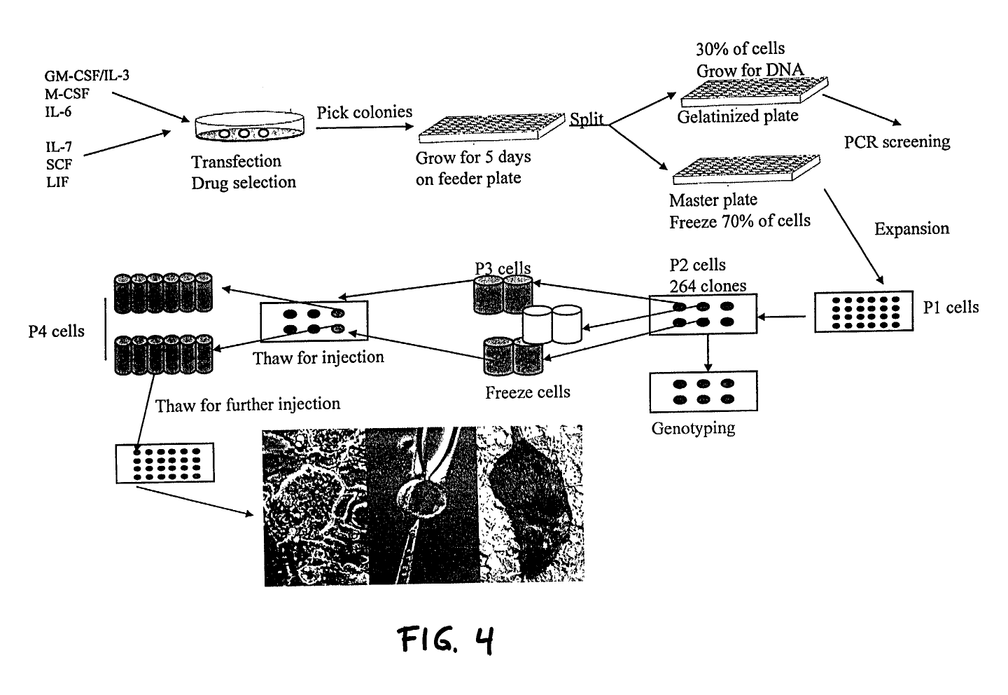

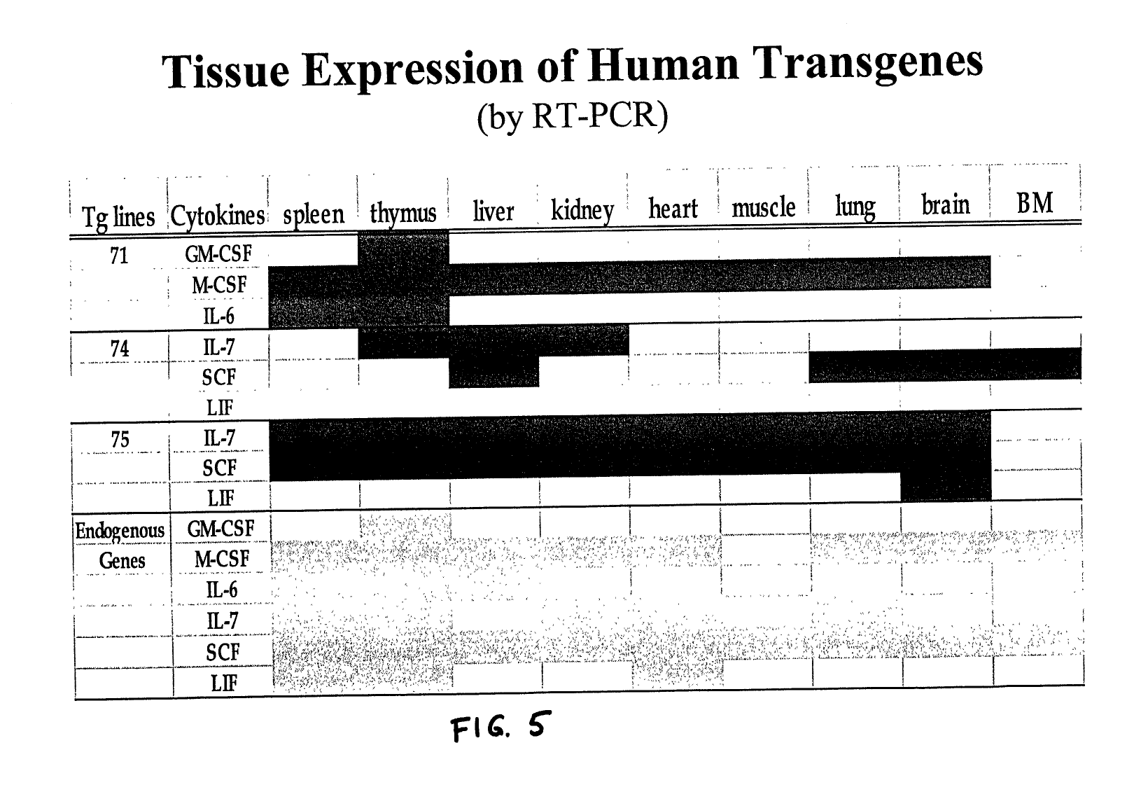

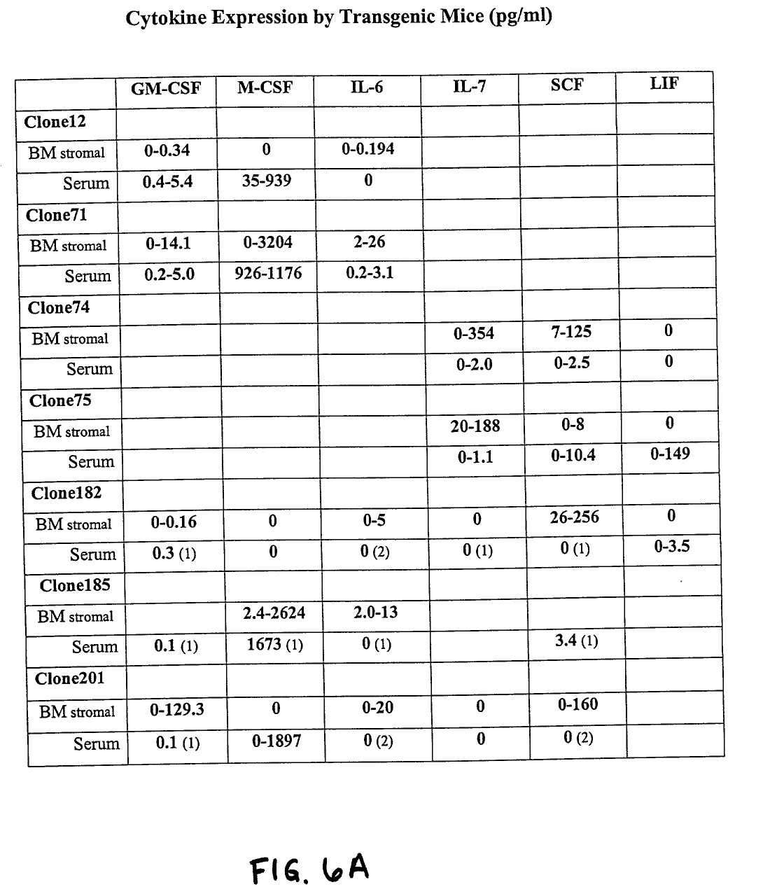

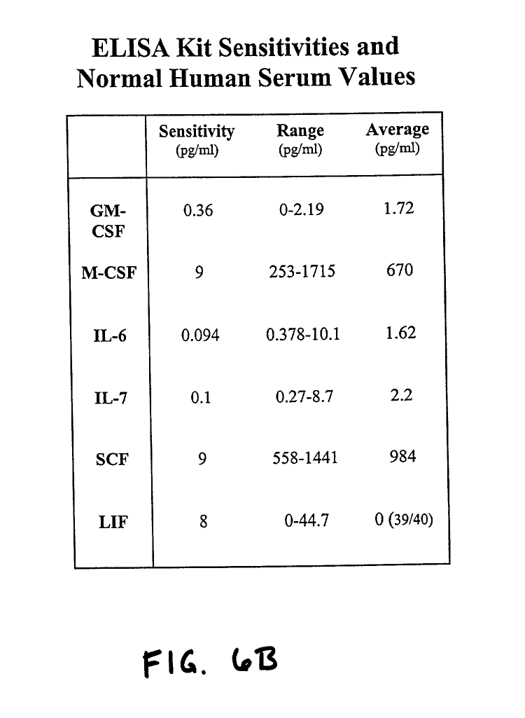

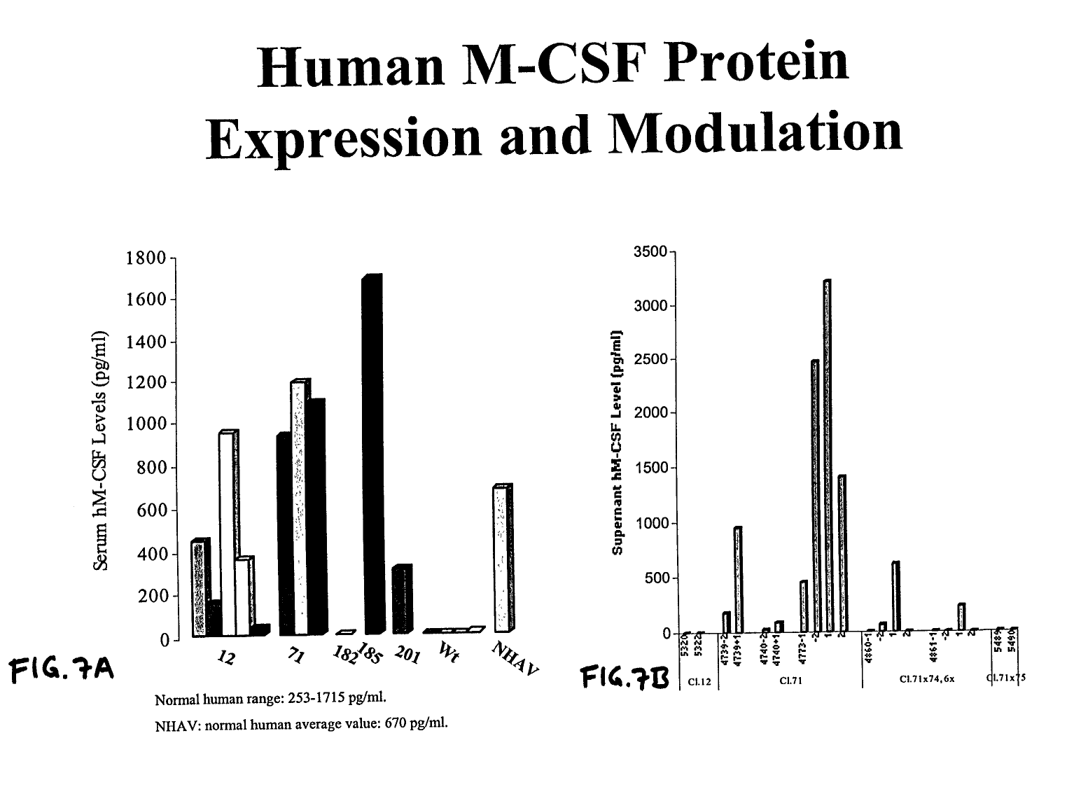

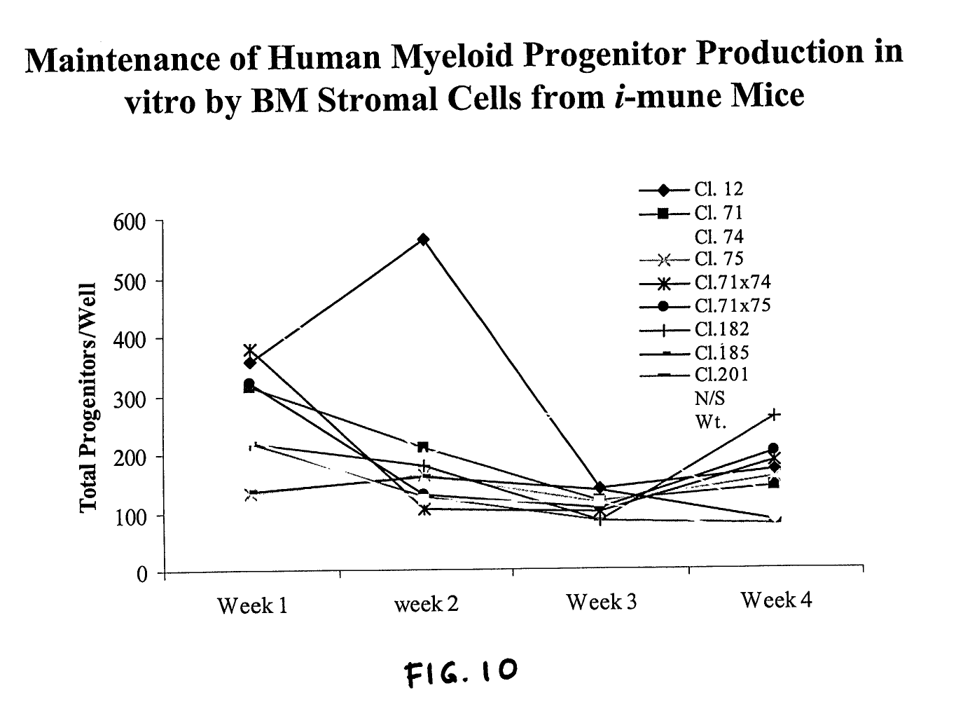

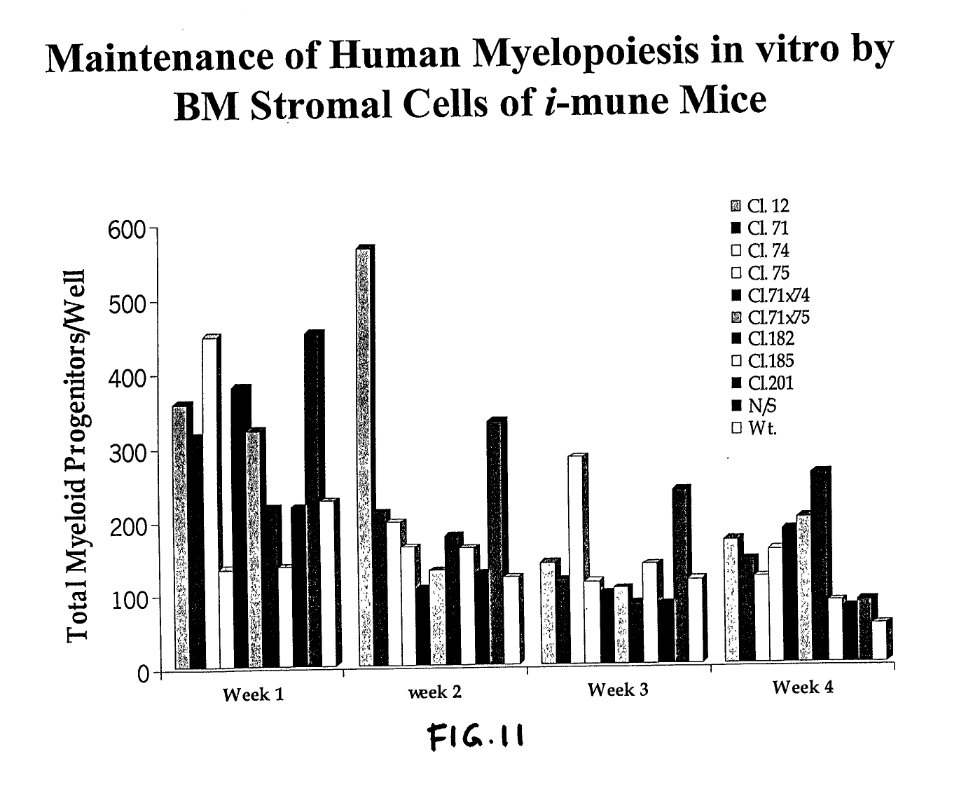

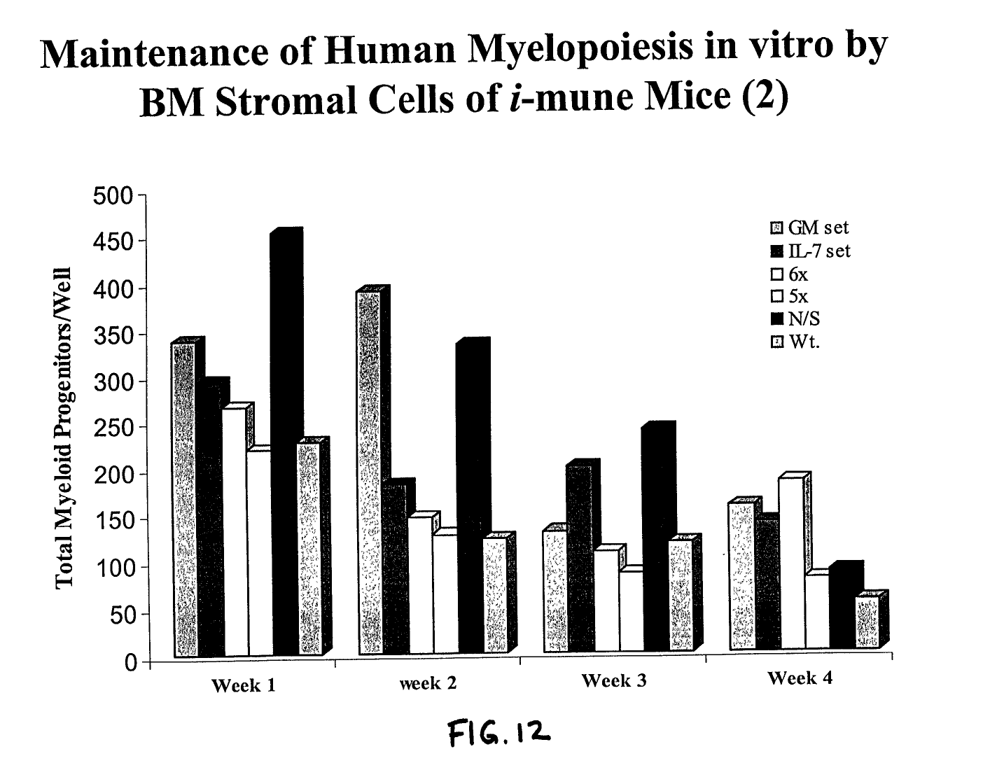

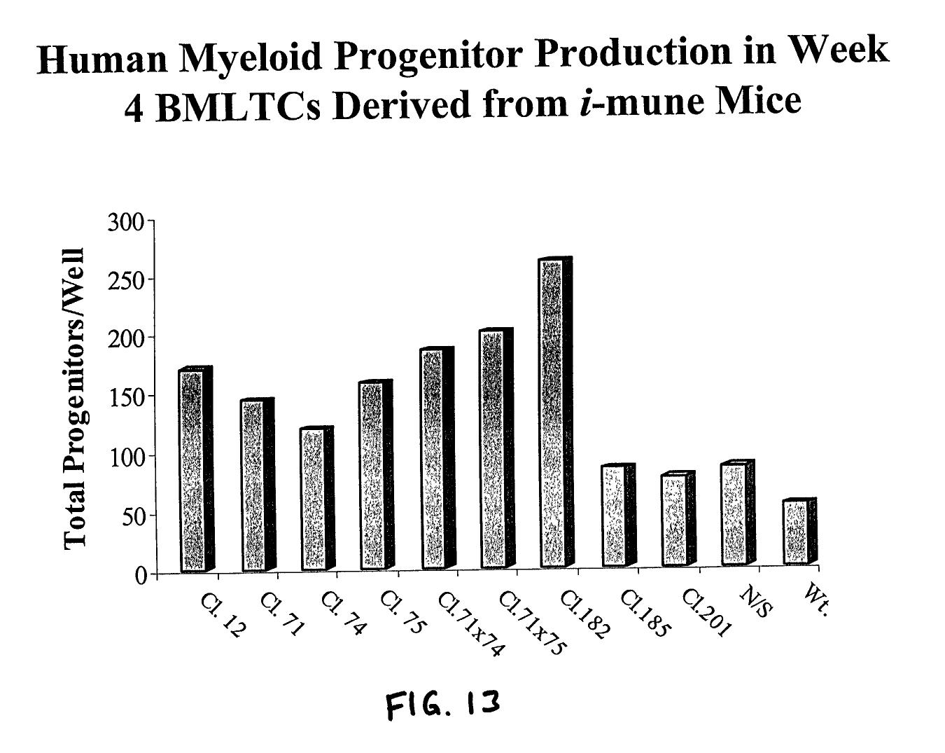

The results, as presented in FIGS. 1-13 show (i) the successful generation of transgenic mouse lines with six human transgenes; (ii) the mRNA expression patterns of these genes are consistent with that of mouse endogenous cytokines; (iii) that all six proteins of transgenes can be detected, five of which (except SCF) have reached normal human serum levels and that their expression level can be modulated by irradiation; (iv) that stromal cells from the cytokine transgenic mice can better support human myelopoiesis in vitro compared to the stromal cells from the non-cytokine transgenic littermates (in the later period of BMLTC (week 4), all cultures derived from transgenic BMCs demonstrated higher myeloid progenitor production compared to the cultures derived from non-cytokine transgenic BMCs); (v) that stromal cultures derived from 7 of 9 cytokine transgenic lines (except for clone 185 and 201) maintain higher human myeloid progenitor production at week 4 of BMLTC compared to stromal cultures derived from NOD/SCID BMC, although NA cell production from NOD/SCID stromal cultures were usually the highest compared to cytokine transgenic and non-cytokine transgenic cultures; and (vi) that human myeloid progenitor production is a better readout for the maintenance of human myelopoiesis in vitro by BM stromal cells of i-mune mice compared to NA cell production.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various publications are cited herein, the disclosures of which are incorporated by reference in their entireties.

What is claimed is:

1. A recipient mouse comprising:
a disruption in both alleles of a gene such that lymphocyte maturation does not occur; and
exogenous transgenes that encode cytokines comprising IL-7, SCF and LIF.

2. A recipient mouse comprising:
a disruption in both alleles of a gene such that lymphocyte maturation does not occur; and
exogenous transgenes that encode cytokines comprising GM-CSF, M-CSF and IL-6.

3. A recipient mouse comprising:
a disruption in both alleles of a gene such that lymphocyte maturation does not occur; and
exogenous transgenes that encode cytokines comprising IL-7, SCF, LIF, GM-CSF, M-CSF and IL-6.

4. The mouse of claims 1-3, wherein the disruption is in a gene that modulates VDJ recombination.

5. The mouse of claim 4, wherein said gene is a RAG gene.

6. The mouse of claims 1-3, wherein the cytokines are human cytokines.

7. A method of making a mouse lacking in mature T and B cells and comprising exogenous cytokines comprising the steps of:
inactivating VDJ recombination; and
introducing transgenes, wherein said transgenes encode human cytokines necessary for support of human cells in the mouse.

8. The method of claim 7, wherein the step of introducing the transgenes is through pronuclear transfer.

9. The method of claim 7, wherein the transgenes are in an embryonic stem cell.

10. The method of claim 7, wherein the step of introducing the transgenes is through breeding said mouse with a mouse that comprises the transgenes.

11. The method of claim 7, wherein the mouse is a RAG-1⁻ or a RAG2⁻ mouse.

12. The method of claim 7 wherein said cytokines comprise IL-7, SCF and LIF.

13. The method of claim 7 wherein said cytokines comprise IL-6, GM-CSF and M-CSF.

14. The method of claim 7 wherein said cytokines comprise IL-7, SCF, LIF, IL-6, GM-CSF and M-CSF.

15. The mouse of claim 1, wherein said mouse further comprises a MHC transgene.

16. The mouse of claim 15, wherein said MHC transgene is a human HLA transgene.

17. A recipient mouse comprising:
a disruption in both alleles of a gene such that lymphocyte maturation does not occur; and
a human transgene comprising a nucleic acid sequence that encodes a MHC Class II DR3 molecule, wherein the transgene comprises naturally linked DRab and DQab alleles.

18. The mouse of claim 17, wherein the disruption is in a gene that modulates VDJ recombination.

19. The mouse of claim 18, wherein the gene is a RAG gene.

20. The mouse of claim 19, wherein said mouse is deficient for murine I-Eα.

21. The mouse of claim 17, wherein the transgene further comprises a human HLA DQ2 gene.

22. A method of making a recipient mouse, said method comprising:
disrupting both alleles of a gene so that lymphocyte maturation does not occur;
inserting a transgene comprising nucleic acid that encodes MHC Class II DR3 and DQ2 molecules, wherein the DRab and DQab alleles are naturally linked; and
inactivating murine I-Eα.

23. The method of claim 22, wherein said disruption is in a gene that modulates VDJ recombination.

24. The method of claim 23, wherein said gene is RAG-2.

25. The method of claim 24, wherein said transgene is in an artificial yeast chromosome.

26. The method of claim 25, wherein the transgene is about 550 kb in length.

27. The method of claim 26, wherein the artificial yeast chromosome is 4D1.

28. A method of making a recipient mouse, said method comprising:
preventing VDJ recombination by mutating both alleles of the RAG-2 gene;
inserting a transgene comprising the Drab and DQab alleles of the MHC Class II DR3 haplotype; and
inactivating murine I-Eα.
